(19) United States
(12) Patent Application Publication
(10) Pub. No.: US 2019/0151304 A1
(43) Pub. Date: May 23, 2019

(54) METHODS AND PHARMACEUTICAL COMPOSITIONS FOR THE TREATMENT OF AUTOIMMUNE INFLAMMATORY

(71) Applicants: INSERM (INSTITUT NATIONAL DE LA SANTÉ ET DE LA RECHERCJAE MÉDICALE, Paris (FR); UNIVERSITÉ DE ROUEN NORMANDIE, Mont-Saint Aignan (FR); UNIVERSITÉ PARIS DIDEROT - PARIS 7, Mont-Saint-Aignan (FR)

(72) Inventors: Yossan-Var TAN, Rouen cedex (FR); Catalina ABAD RABAT, Rouen cedex (FR); Thierry VOISIN, Paris (FR); Alain COUVINEAU, Paris Cedex 18 (FR)

(21) Appl. No.: 16/300,328

(22) PCT Filed: May 9, 2017
(86) PCT No.: PCT/EP2017/061076

§ 371 Date: Nov. 9, 2018

(30) Foreign Application Priority Data
May 10, 2016 (EP) 16305542.9

(51) Int. Cl. A61K 31/47; A61P 37/06

(52) U.S. Cl. CPC A61K 31/47; A61P 37/06

(57) ABSTRACT

The present invention relates to methods and pharmaceutical compositions for the treatment of autoimmune inflammatory diseases. The inventors showed that orexin receptor antagonists have anti-inflammatory properties. Indeed, these compounds are antagonist for OX1R-mediated calcium mobilization but a full agonist for OX1R-mediated mitochondrial apoptosis, which is the mechanism involved in the improvement of resolution of inflammation observed in the models of colitis, multiple sclerosis and pancreatitis. In particular, the present invention relates to a method of treating an autoimmune inflammatory disease in a subject in need thereof comprising administering to the subject a therapeutically effective amount of at least one OX1R antagonist.

### FIELD OF THE INVENTION

The present invention relates to methods and pharmaceutical compositions for the treatment of autoimmune inflammatory diseases.

### BACKGROUND OF THE INVENTION

Inflammation is a coordinated process designed by evolution to eliminate pathogens and enable healing. However, this is carefully orchestrated in the sense that when it is no longer necessary, it must be actively terminated to avoid tissue damage and/or auto-immunity. In this line, if the activities of the pro-inflammatory IFN-γ (or Th1) and IL-17 (or Th17) producing T helper cells are not efficiently modulated after host defense, these T cell subsets contribute to autoimmune inflammatory conditions such as multiple sclerosis (MS), inflammatory bowel disease (IBD) and chronic pancreatitis. For instance, inflammatory bowel disease (IBD) is a chronic inflammatory disorder of the gastrointestinal tract that comprises Crohn's disease (CD) and ulcerative colitis (UC). For a long time, CD was considered to be Th1-driven and UC Th2-driven, but more recently Th17 cells may participate in their pathogeneses. In the same way, chronic pancreatitis which is a progressive inflammatory disease of the pancreas leading to inflammation and fibrosis associated to the exocrine and endocrine insufficiency, is characterized by a predominance of Th1 response.

Multiple sclerosis is a chronic demyelinating disease of the central nervous system. Despite its complex pathogenesis, evidence supports an autoimmune component of the disease driving chronic inflammatory processes in the spinal cord and brain. Although it was classically considered that the nervous and immune systems were independent from each other, it is now known that they interact through common mediators and receptors. In this sense, the list of neuropeptides that exert immunomodulatory properties is continuously growing.

Orexin A and orexin B (also known as hypocretin 1 and hypocretin 2, respectively), are two neuropeptides derived from a common precursor polypeptide, which were initially identified as endogenous ligands for two orphan G protein-coupled receptors, OX1R and OX2R. Originally discovered in the hypothalamus, they are mainly known for their ability to regulate sleep and arousal states, appetite and feeding, gastrointestinal mobility and energy homeostasis. The potential involvement of orexin receptors in the immune system has been barely investigated.

### SUMMARY OF THE INVENTION

The present invention relates to methods and pharmaceutical compositions for the treatment of autoimmune inflammatory diseases such as multiple sclerosis. In particular, the present invention is defined by the claims.

### DETAILED DESCRIPTION OF THE INVENTION

Surprisingly, the inventors showed that orexin receptor antagonists have anti-inflammatory properties. Indeed, these compounds are antagonist for OX1R-mediated calcium mobilization but a full agonist for OX1R-mediated mitochondrial apoptosis, which is the mechanism involved in the improvement of resolution of inflammation observed in the models of colitis, multiple sclerosis and pancreatitis.

A first aspect of the present invention relates to a method of treating an autoimmune inflammatory disease in a subject in need thereof comprising administering to the subject a therapeutically effective amount of at least one OX1R antagonist.

As used herein, the term “subject” denotes a mammal, such as a rodent, a feline, a canine, and a primate. Preferably, a subject according to the invention is a human.

As used herein, the expression “autoimmune inflammatory disease” is used herein in the broadest sense and includes all diseases and pathological conditions where the pathogenesis of which involves abnormalities of Th1 and Th17 cells, in particulate accumulation of Th1 and Th17 cells in organs. As used herein, the term “Th17 cells” has its general meaning in the art and refers to a subset of T helper cells producing interleukin 17 (IL-17). “A brief history of T(H)17, the first major revision in the T(H)1/T(H)2 hypothesis of T cell-mediated tissue damage”. Nat. Med. 13 (2): 139-145, 2007). The term “IL-17” has its general meaning in the art and refers to the interleukin-17A protein. Typically, Th17 cells are characterized by classical expression of Th cell markers at their cell surface such as CD4, and by the expression of IL17. Typically, as referenced herein, a Th17 cell is a IL-17+ cell. As used herein, the term “Th1 cell” mean a type-1 helper T cell characterized by classical expression of CD4 and its ability to produce high levels of the proinflammatory cytokine IFNγ.

In particular, the above-mentioned autoimmune inflammatory diseases may be one or more selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, the method of the present invention is particularly suitable for the treatment of multiple sclerosis.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses the prophylactic treatment. As used herein, the term “prevent” refers to the reduction in the risk of acquiring or developing a given condition, or the reduction or inhibition of the recurrence or said condition in a subject who is not ill, but who has been or may be near a subject with the disease.

As used herein, the term “OX1R” has its general meaning in the art and refers to the 7-transmembrane spanning receptor OX1R for orexins. An exemplary amino acid sequence of OX1R is shown as SEQ ID NO:1.

SEQ ID NO: 1: human orexin receptor-1 OX1R
MEPSATPGAQ MGVPPGSREP SPVPPDYEDE FLRYLWRDYL
YPKQYEWVLI AAYVAVFVVA LVGNTLVCLA VWRNHHMRTV
TNYFIVNLSL ADVLVTAICL PASLLVDITE SWLFGHALCK
VIPYLQAVSV SVAVLTLSFI ALDRWYAICH PLLFKSTARR
ARGSILGIWA VSLAIMVPQA AVMECSSVLP ELANRTRLFS
VCDERWADDL YPKIYHSCFF IVTYLAPLGL MAMAYFQIFR
KLWGRQIPGT TSALVRNWKR PSDQLGDLEQ GLSGEPQPRG
RAFLAEVKQM RARRKTAKML MVVLLVFALC YLPISVLNVL
KRVFGMFRQA SDREAVYACF TFSHWLVYAN SAANPIIYNF
LSGKFREQFK AAFSCCLPGL GPCGSLKAPS PRSSASHKSL
SLQSRCSISK ISEHVVLTSV TTVLP

As used herein the term “OX1R antagonist” has its general meaning in the art and refers to any compound that is able to inhibit the calcium-dependent signalling pathway induced by Orexin. It is known that binding of the orexin to its receptor triggers an influx of calcium, which is coupled to activation of Erk. The receptors also couple to a phospholipase C (PLC)-mediated pathway that releases intracellular calcium stores. The utility of the compounds in accordance with the present invention as orexin receptor OX1R antagonists may be readily determined without undue experimentation by methodology well known in the art, including the “FLIPR Ca2+ Flux Assay” (Okumura et al, Biochem. Biophys. Res. Comm. 280:976-981, 2001). In a typical experiment the OX1 receptor antagonistic activity of the compounds of the present invention was determined in accordance with the following experimental method. For intracellular calcium measurements, Chinese hamster ovary (CHO) cells expressing the rat orexin-1 receptor are grown in Iscove's modified DMEM containing 2 mM L-glutamine, 0.5 g/ml G418, 1% hypoxanthine-thymidine supplement, 100 U/ml penicillin, 100 ug/ml streptomycin and 10% heat-inactivated fetal calf serum (FCS). The cells are seeded at 20,000 cells/well into Becton-Dickinson black 384-well clear bottom sterile plates coated with poly-D-lysine. All reagents were from GIBCO-Invitrogen Corp. The seeded plates are incubated overnight at 37° C. and 5% C02. Ala-6,12 human orexin-A as the agonist is prepared as a 1 mM stock solution in 1% bovine serum albumin (BSA) and diluted in assay buffer (HBSS containing 20 mM HEPES, 0.1% BSA and 2.5 mM probenecid, pH7.4) for use in the assay at a final concentration of 70 pM. Test compounds are prepared as 10 mM stock solution in DMSO, then diluted in 384-well plates, first in DMSO, then assay buffer. On the day of the assay, cells are washed 3 times with 100 ul assay buffer and then incubated for 60 min (37° C., 5% C02) in 60 ul assay buffer containing 1 uM Fluo-4AM ester, 0.02% pluronic acid, and 1% BSA. The dye loading solution is then aspirated and cells are washed 3 times with 100 ul assay buffer. 30 ul of that same buffer is left in each well. Within the Fluorescent Imaging Plate Reader (FLIPR, Molecular Devices), test compounds are added to the plate in a volume of 25 ul, incubated for 5 min and finally 25 ul of agonist is added. Fluorescence is measured for each well at 1 second intervals for 5 minutes and the height of each fluorescence peak is compared to the height of the fluorescence peak induced by 70 pM Ala-6,12 orexin-A with buffer in place of antagonist. For each antagonist, IC50 value (the concentration of compound needed to inhibit 50% of the agonist response) is determined. Alternatively, compound potency can be assessed by a radioligand binding assay (described in Bergman et. al. Bioorg. Med. Chem. Lett. 2008, 18, 1425-1430) in which the inhibition constant is determined in membranes prepared from CHO cells expressing the OX1 receptor. The intrinsic orexin receptor antagonist activity of a compound which may be used in the present invention may be determined by these assays.

In one embodiment, the OX1R antagonist is a small organic molecule. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more in particular up to 2000 Da, and most in particular up to about 1000 Da.

OX1R antagonists are well known to the skilled person who may easily identify such antagonists from the following literature:
Boss C, Roch-Brisbare C, Steiner M A, Treiber A, Dietrich H, Jenck F, von Raumer M, Sifferlen T, Brotschi C, Heidmann B, Williams J T, Aissaoui H, Siegrist R, Gatfield J. Structure-Activity Relationship, Biological, and Pharmacological Characterization of the Proline Sulfonamide ACT-462206: a Potent, Brain-Penetrant Dual Orexin 1/Orexin 2 Receptor Antagonist. Chem Med Chem. 2014 Aug. 21.
Christopher J A. Orexin receptor antagonists. Pharm Pat Anal. 2012 July; 1(3):329-46.
Coleman P J, Schreier J D, Cox C D, Breslin M J, Whitman D B, Bogusky M J, McGaughey G B, Bednar R A, Lemaire W, Doran S M, Fox S V, Garson S L, Gotter A L, Harrell C M, Reiss D R, Cabalu T D, Cui D, Prueksaritanont T, Stevens J, Tannenbaum P L, Ball R G, Stellabott J, Young S D, Hartman G D, Winrow C J, Renger J J. Discovery of [(2R,5R)-5-{[(5-fluoropyridin-2-yl)oxy]methyl}-2-methylpiperidin-1-yl][5-methyl-2-(pyrimidin-2-yl)phenyl]methanone (MK-6096): a dual orexin receptor antagonist with potent sleep-promoting properties. Chem Med Chem. 2012 Mar. 5; 7(3):415-24, 337.
Cox C D, Breslin M J, Whitman D B, Schreier J D, McGaughey G B, Bogusky M J, Roecker A J, Mercer S P, Bednar R A, Lemaire W, Bruno J G, Reiss D R, Harrell C M, Murphy K L, Garson S L, Doran S M, Prueksaritanont T, Anderson W B, Tang C, Roller S, Cabalu T D, Cui D, Hartman G D, Young S D, Koblan K S, Winrow C J, Renger J J, Coleman P J. Discovery of the dual orexin receptor antagonist [(7R)-4-(5-chloro-1,3-benzoxazol-2-yl)-7-methyl-1,4-diazepan-1-yl][5-methyl-2-(2H-1,2,3-triazol-2-yl)phenyl]methanone (MK-4305) for the treatment of insomnia. J Med Chem. 2010 Jul. 22; 53(14):5320-32.
Jiaqiang Cai, Fiona E Cooke, Bradley S Sherborne Antagonists of the orexin receptors Expert Opinion on Therapeutic Patents May 2006, Vol. 16, No. 5, Pages 631-646: 631-646.
John A Christopher, Miles S Congreve Treatment and prevention of various therapeutic conditions using OX receptor antagonistic activity (WO2012081692) Expert Opinion on Therapeutic Patents February 2013, Vol. 23, No. 2, Pages 273-277: 273-277.
Langmead C J, Jerman J C, Brough S J, Scott C, Porter R A, Herdon H J. Characterisation of the binding of [3H]-SB-674042, a novel nonpeptide antagonist, to the human orexin-1 receptor. Br J Pharmacol. 2004 January; 141(2):340-6. Epub 2003 Dec 22.
Paul J Coleman, John J Renger Orexin receptor antagonists: a review of promising compounds patented since 2006 Expert Opinion on Therapeutic Patents March 2010, Vol. 20, No. 3, Pages 307-324: 307-324.
Perrey D A, German N A, Gilmour B P, Li J X, Harris D L, Thomas B F, Zhang Y. Substituted tetrahydroisoquinolines as selective antagonists for the orexin 1 receptor. J Med Chem. 2013 Sep. 12; 56(17):6901-16.
Perrey D A, Gilmour B P, Runyon S P, Thomas B F, Zhang Y. Diaryl urea analogues of SB-334867 as orexin-1 receptor antagonists. Bioorg Med Chem Lett. 2011 May 15; 21(10):2980-5.
Porter R A, Chan W N, Coulton S, Johns A, Hadley M S, Widdowson K, Jerman J C, Brough S J, Coldwell M, Smart D, Jewitt F, Jeffrey P, Austin N. 1,3-Biarylureas as selective non-peptide antagonists of the orexin-1 receptor. Bioorg Med Chem Lett. 2001 Jul. 23; 11(14):1907-10.
Roecker A J, Coleman P J (2008). “Orexin receptor antagonists: medicinal chemistry and therapeutic potential”. Curr Top Med Chem 8 (11): 977-87.
Roecker A J, Coleman P J. Orexin receptor antagonists: medicinal chemistry and therapeutic potential. Curr Top Med Chem. 2008; 8(11):977-87.
Roecker A J, Mercer S P, Harrell C M, Garson S L, Fox S V, Gotter A L, Prueksaritanont T, Cabalu T D, Cui D, Lemaire W, Winrow C J, Renger J J, Coleman P J. Discovery of dual orexin receptor antagonists with rat sleep efficacy enabled by expansion of the acetonitrile-assisted/diphosgene-mediated 2,4-dichloropyrimidine synthesis. Bioorg Med Chem Lett. 2014 May 1; 24(9):2079-85.
Smart D, Sabido-David C, Brough S J, Jewitt F, Johns A, Porter R A, Jerman J C. SB-334867-A: the first selective orexin-1 receptor antagonist. Br J Pharmacol. 2001 March; 132(6):1179-82.
Whitman D B, Cox C D, Breslin M J, Brashear K M, Schreier J D, Bogusky M J, Bednar R A, Lemaire W, Bruno J G, Hartman G D, Reiss D R, Harrell C M, Kraus R L, Li Y, Garson S L, Doran S M, Prueksaritanont T, Li C, Winrow C J, Koblan K S, Renger J J, Coleman P J. Discovery of a potent, CNS-penetrant orexin receptor antagonist based on an n,n-disubstituted-1,4-diazepane scaffold that promotes sleep in rats. Chem Med Chem. 2009 July; 4(7):1069-74.
Yoshida Y, Terauchi T, Naoe Y, Kazuta Y, Ozaki F, Beuckmann C T, Nakagawa M, Suzuki M, Kushida I, Takenaka O, Ueno T, Yonaga M. Design, synthesis, and structure-activity relationships of a series of novel N-aryl-2-phenylcyclopropanecarboxamide that are potent and orally active orexin receptor antagonists. Bioorg Med Chem. 2014 Sep. 8. pii: S0968-0896(14)00630-0.

Other examples of OX1R antagonists are also described in the following patent publications:

EP0849361

US20080132490

US20090163485

U.S. Pat No. 6,309,854

WO 2014099698

WO00047576

WO00047577

WO00047580

WO01000787

WO01068609

WO01085693

WO01096302

WO02044172

WO02051232

WO02051838

WO02089800

WO02090355

WO03002559

WO03002561

WO03032991

WO03037847

WO03041711

WO03051368

WO03051871

WO03051872

WO03051873

WO19909024

WO19958533

WO2003002561

WO2003051872

WO2004004733

WO2004026866

WO2004033418

WO2004041791

WO2004041807

WO2004041816

WO2004052876

WO2004085403

WO2004096780

WO2005060959

WO2005075458

WO2005118548

WO2005118548

WO2006067224

WO2006110626

WO2006127550

WO2007019234

WO2007025069

WO2007061763

WO2007085718

WO2007122591

WO2007126934

WO2007126935

WO2008008517

WO2008008518

WO2008008551

WO2008020405

WO2008026149

WO2008038251

WO2008065626

WO2008078291

WO2008081399

WO2008107335

WO2008110488

WO2008117241

WO2008143856

WO2008147518

WO2008150364

WO2009003993

WO2009003997

WO2009004585

WO2009011775

WO2009016087

WO2009016564

WO2009020642

WO2009022311

WO2009034133

WO2009058238

WO2009079637

WO2011053522

WO2013059163

WO2013059222

WO2013062857

WO2013062858

WO2014066196

WO2014085208

WO2014099696

and WO2014099697.

In some embodiments, the OX1R antagonist of the present invention is SB408124 which is:

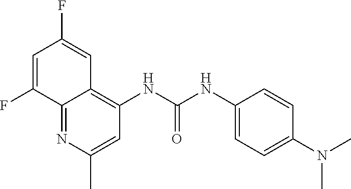 [SMILES: Cc1cc(NC(=O)Nc2ccc(N(C)C)cc2)c2cc(F)cc(F)c2n1]

In some embodiments, the OX1R antagonist of the present invention is selected from the group consisting of:

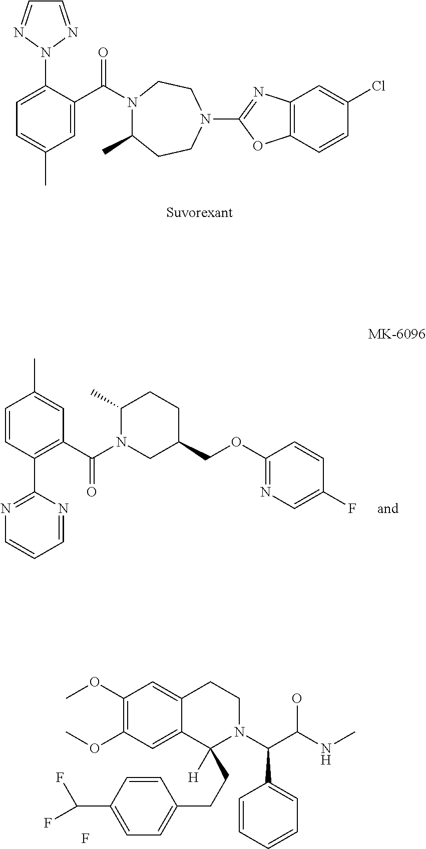

In some embodiments, the OX1R antagonist of the present invention is selected from the group consisting of:

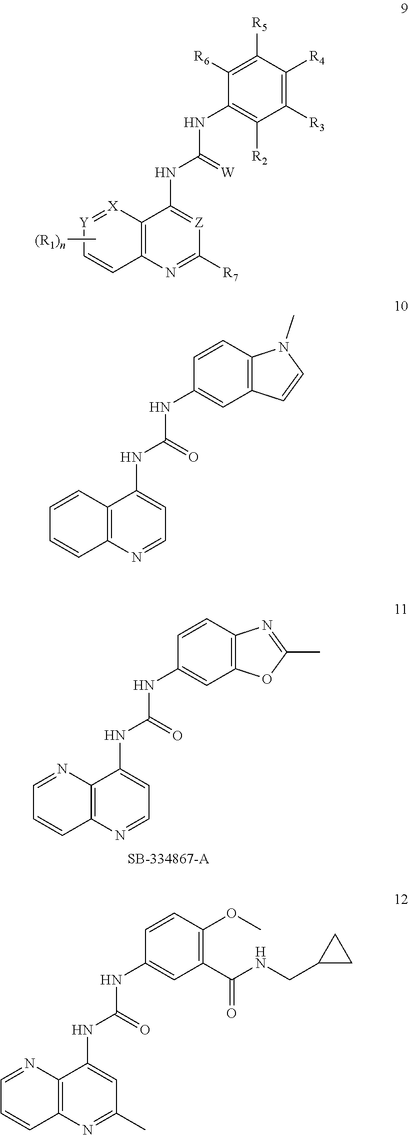

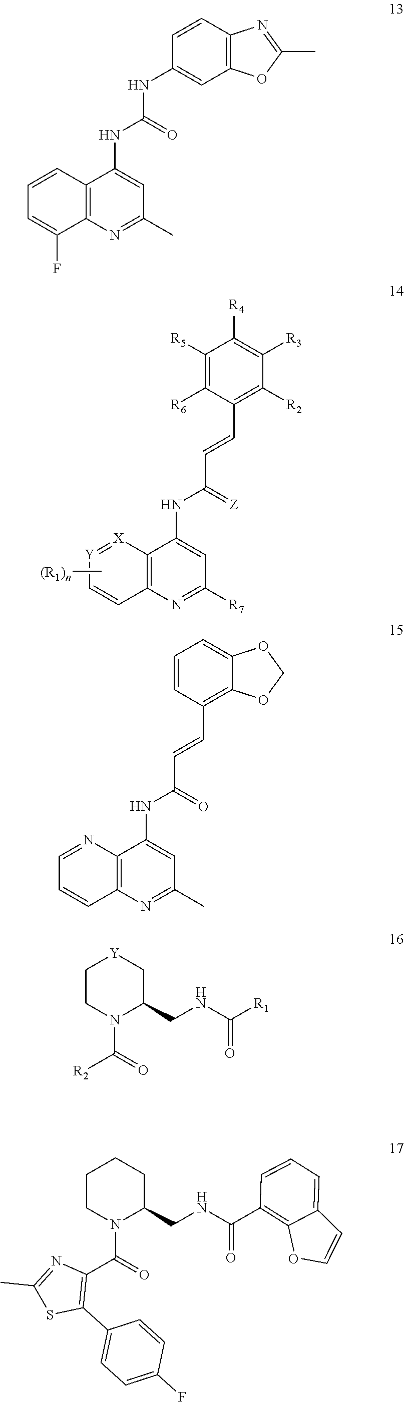

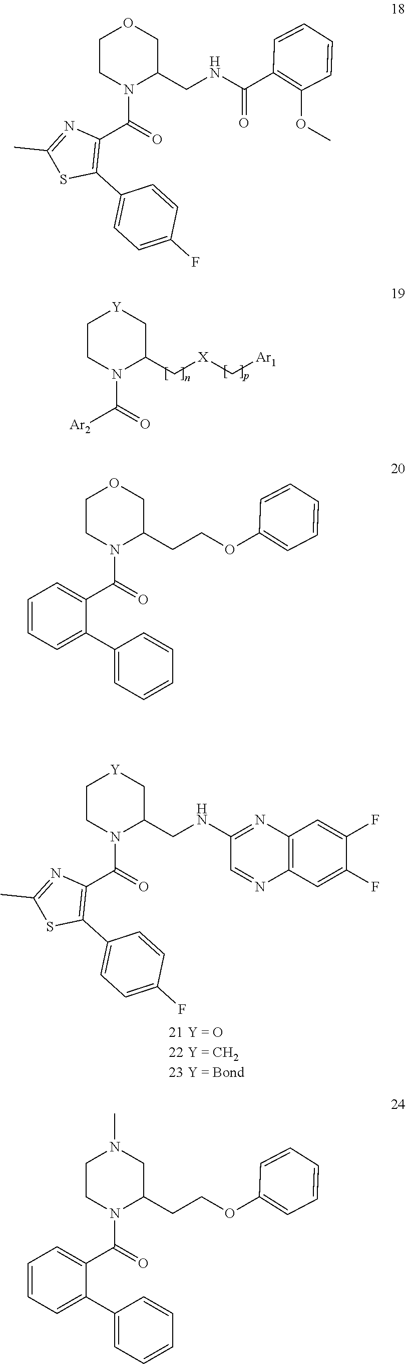

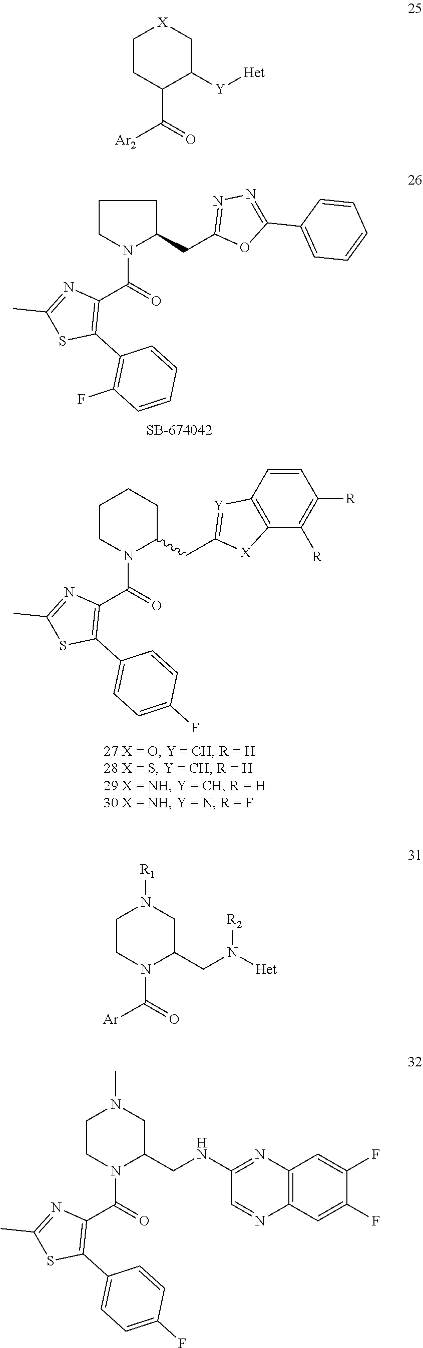

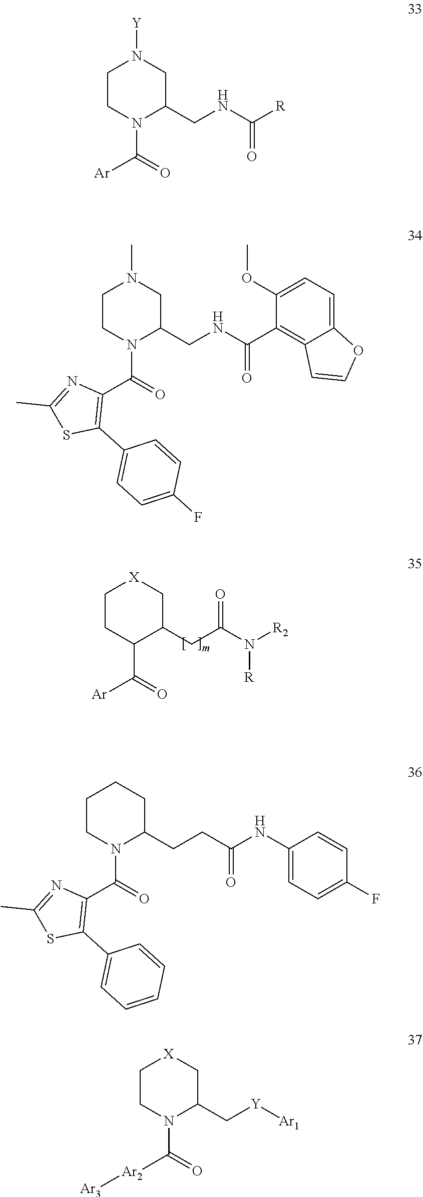

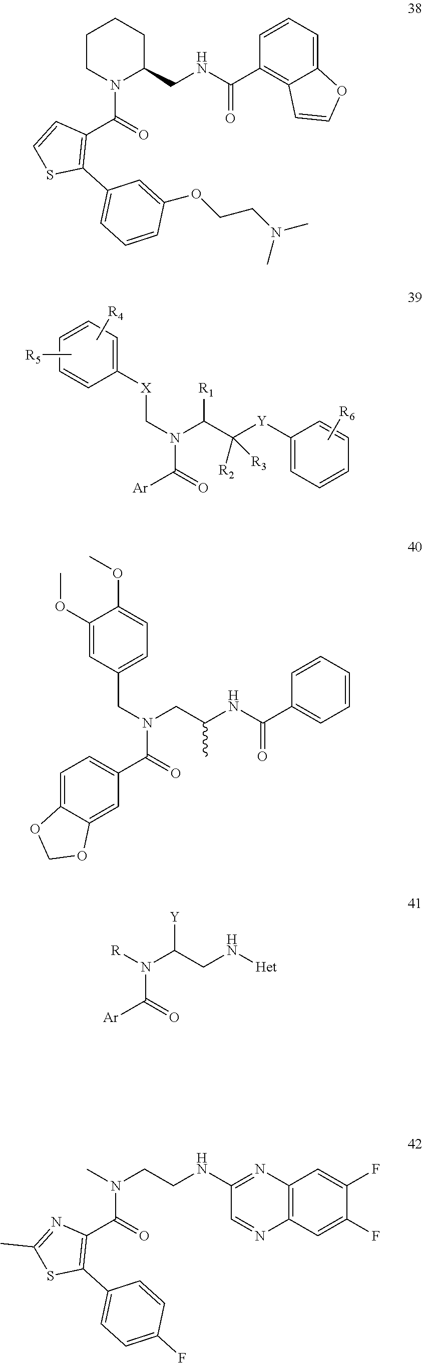

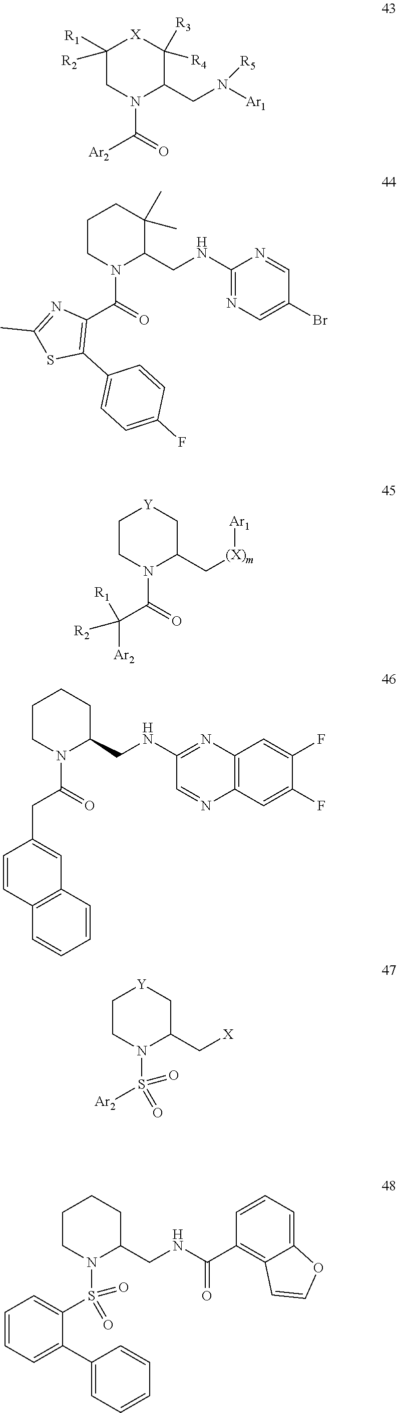

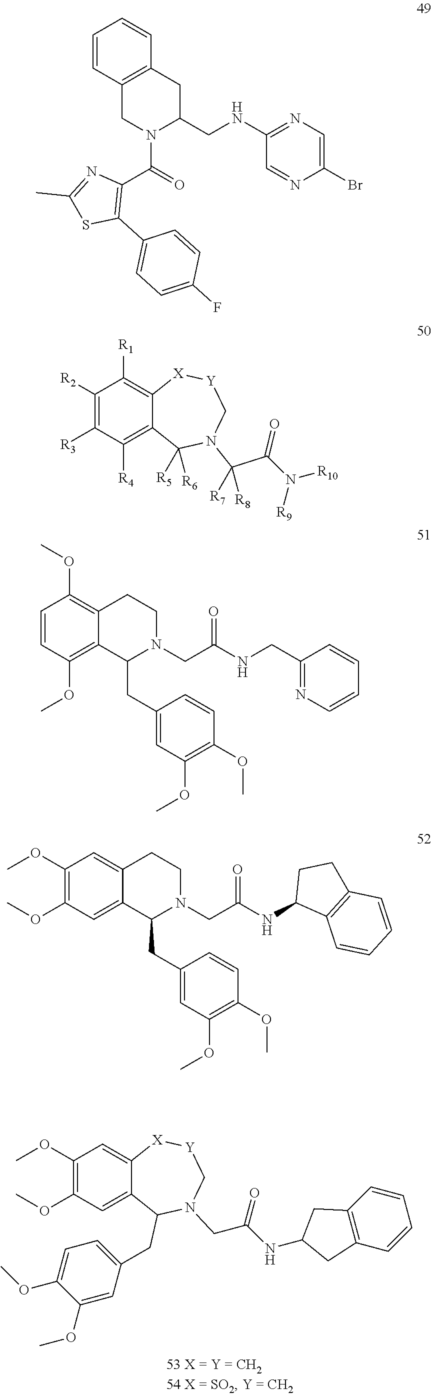

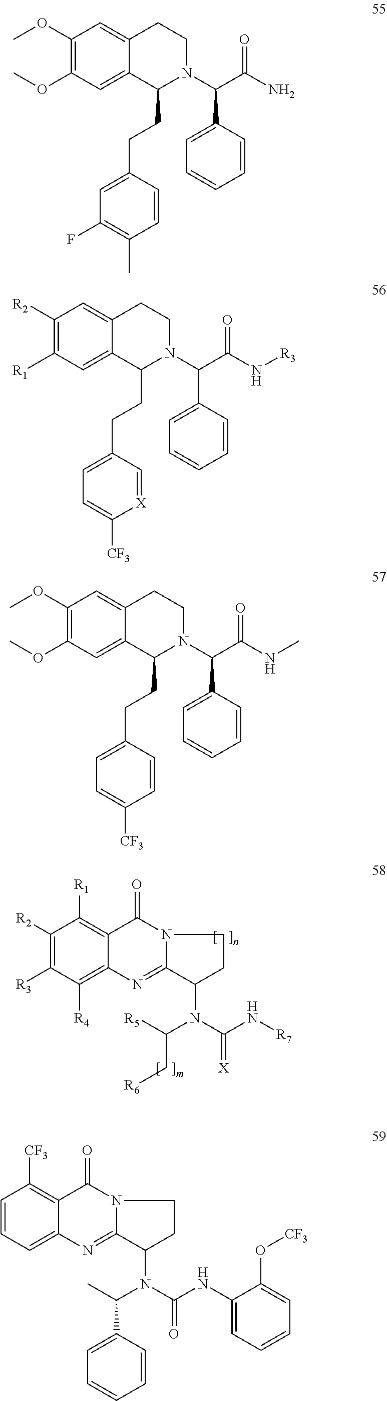

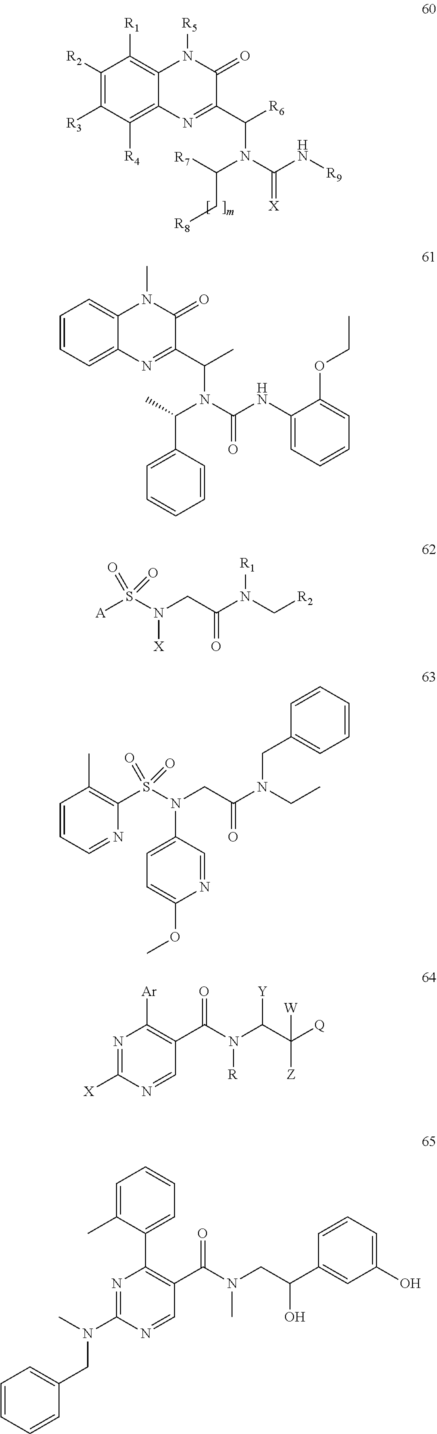

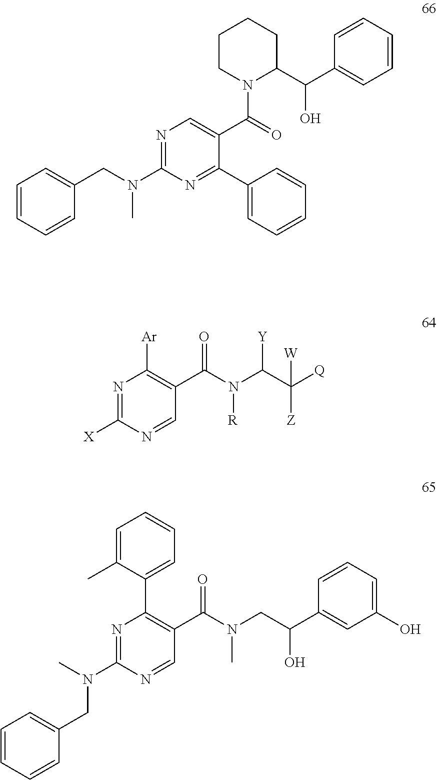

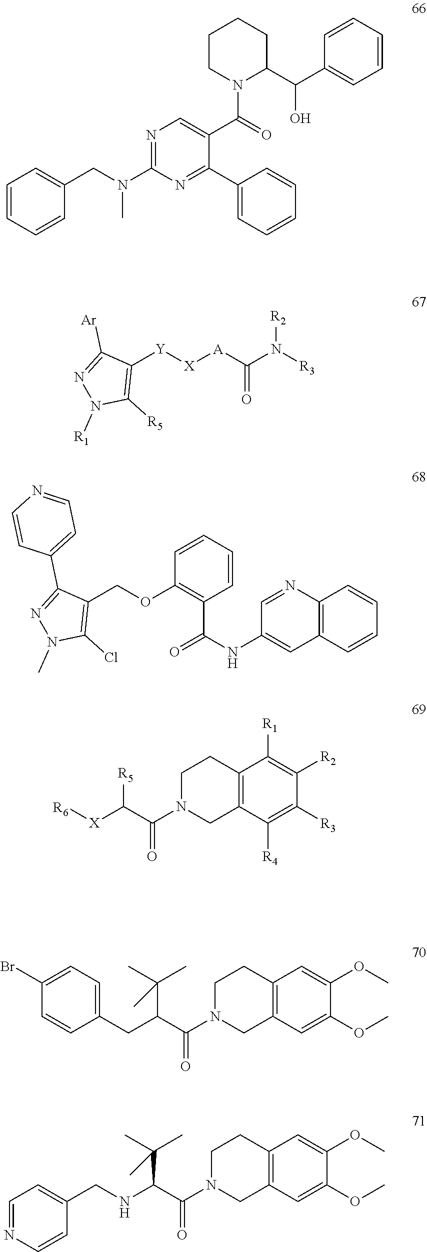

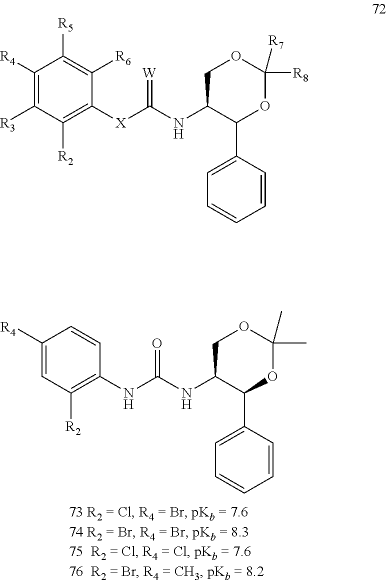

In some embodiments, the OX1R antagonist of the present invention is selected from the group consisting of:

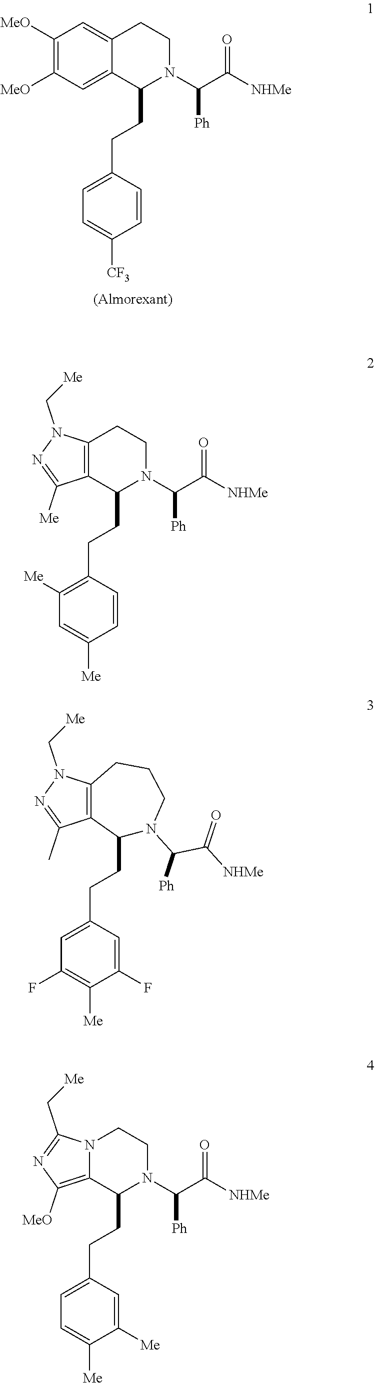

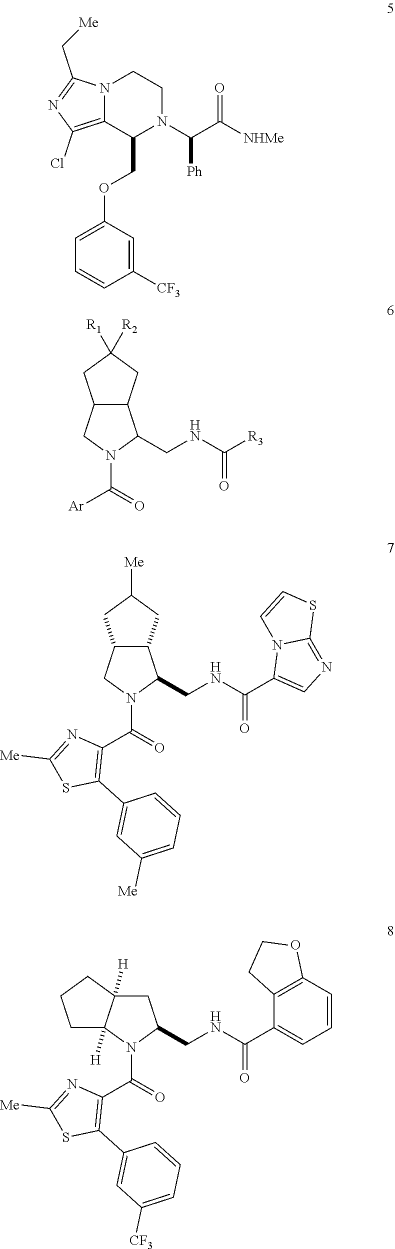

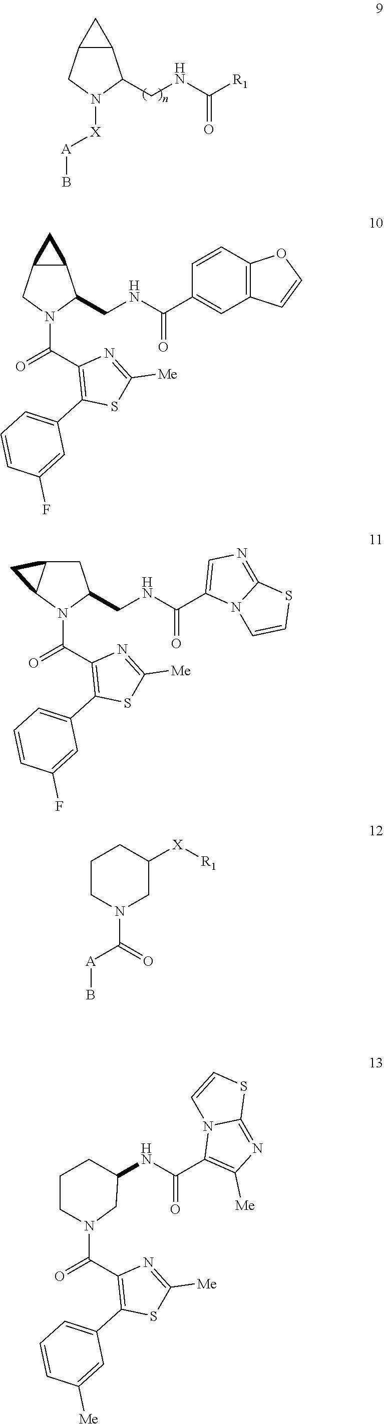

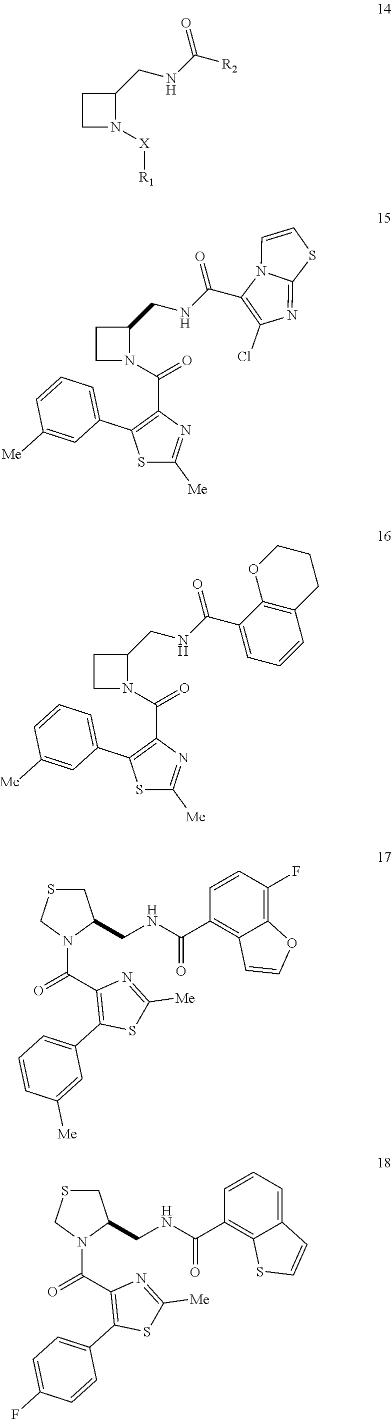

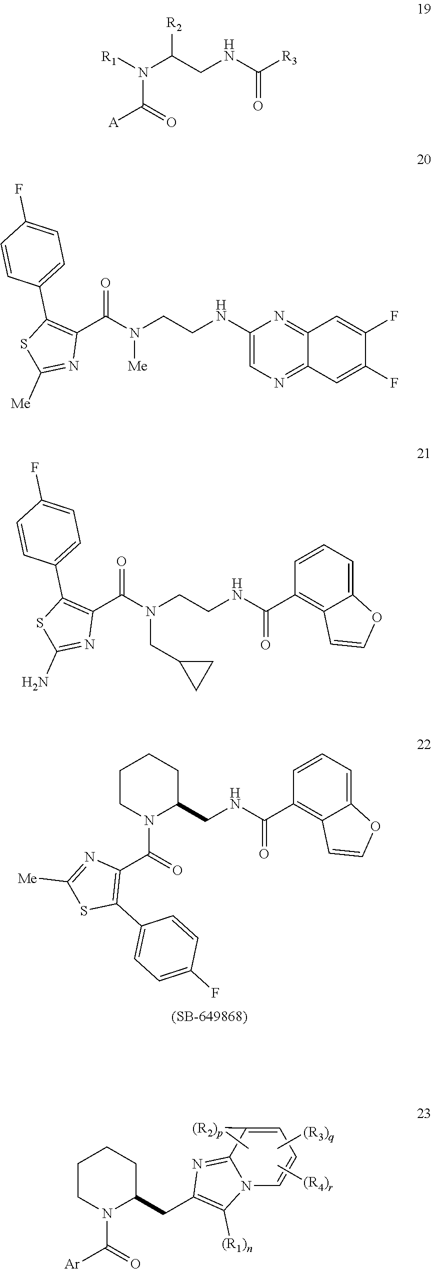

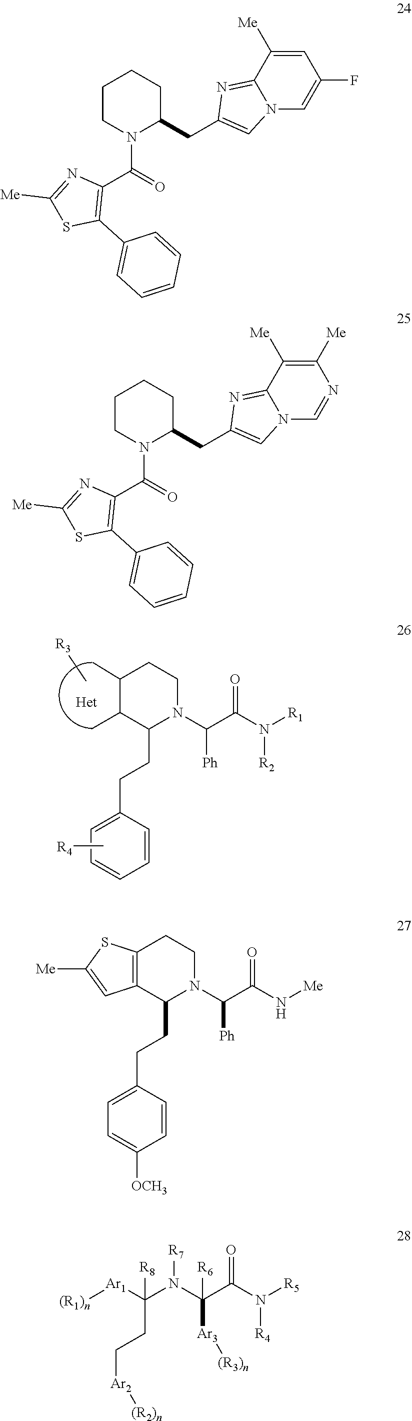

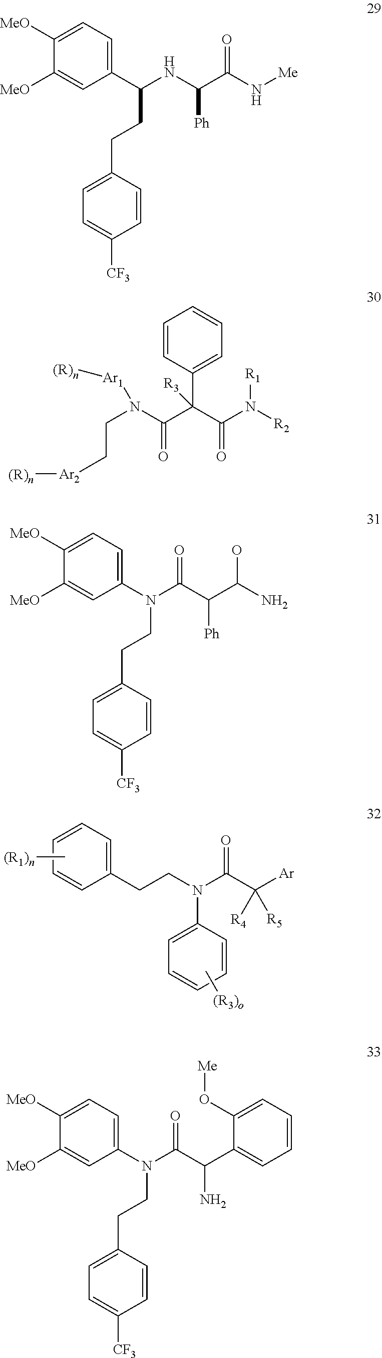

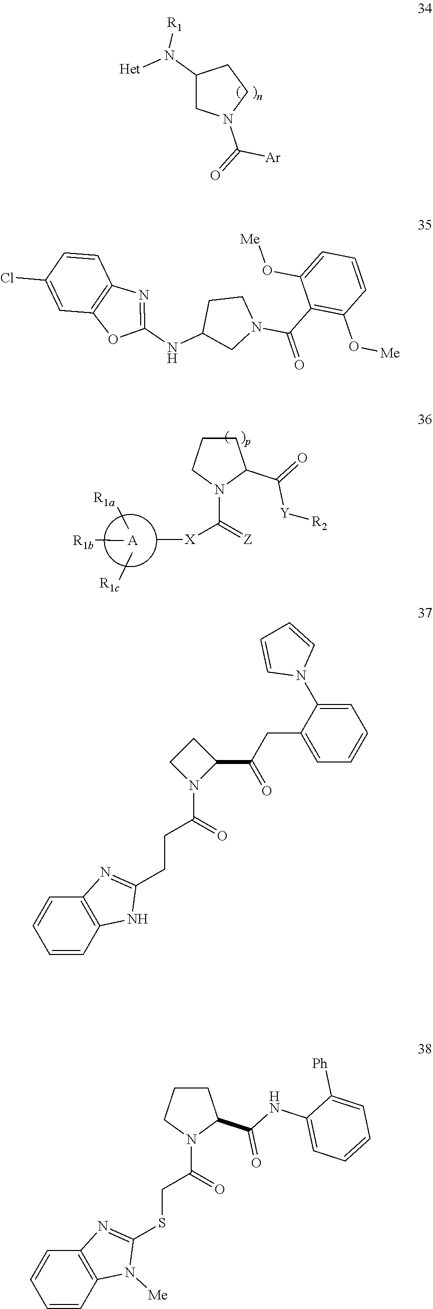

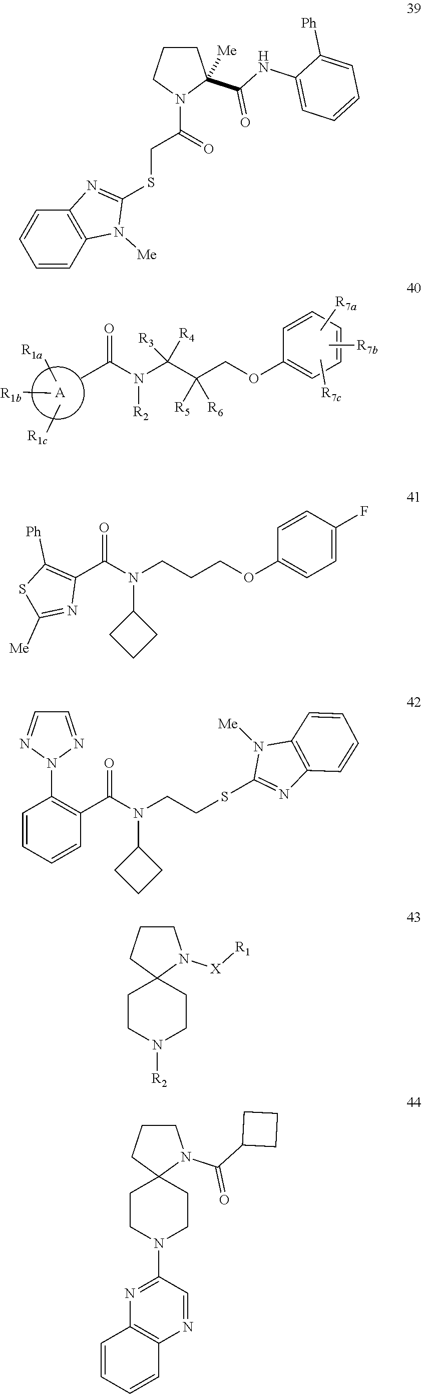

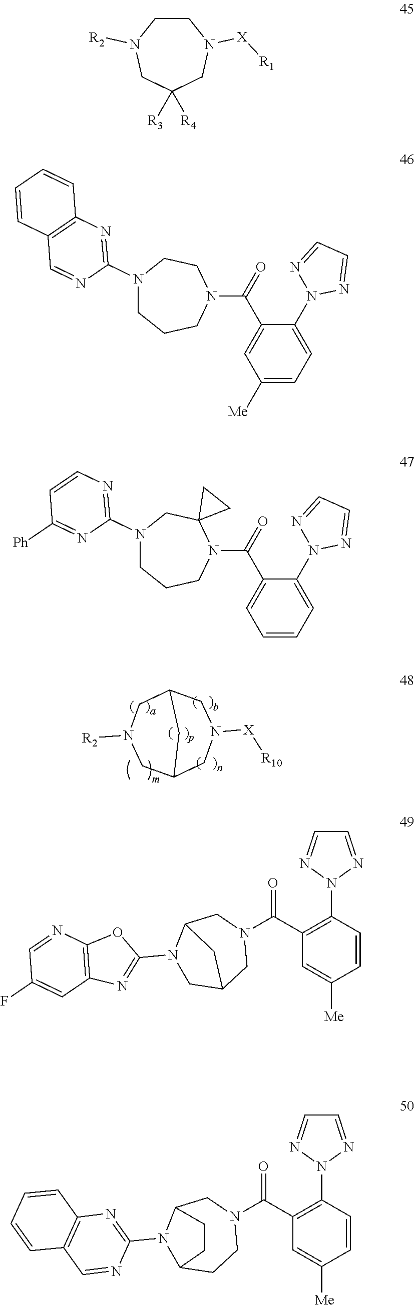

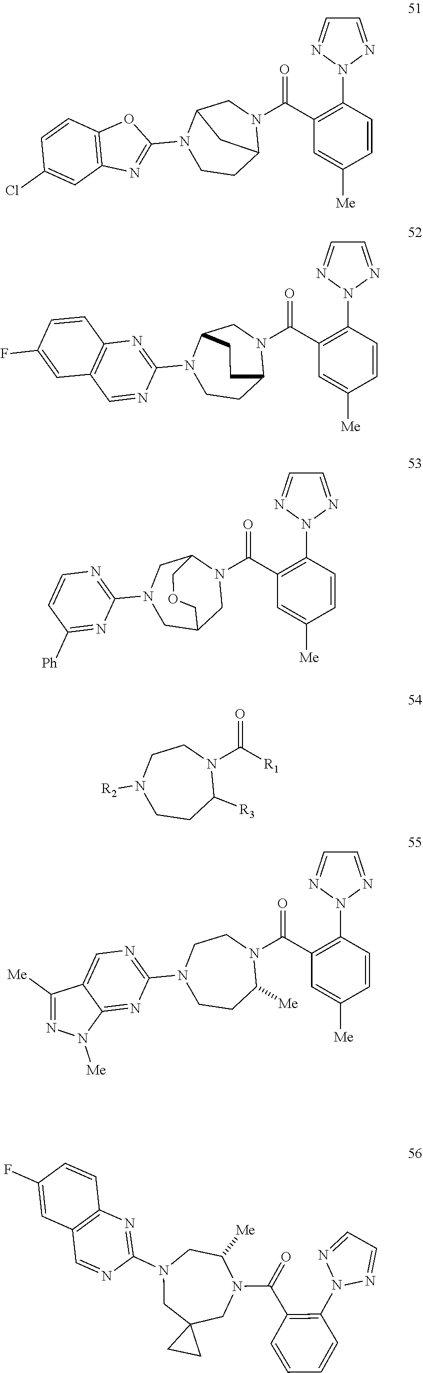

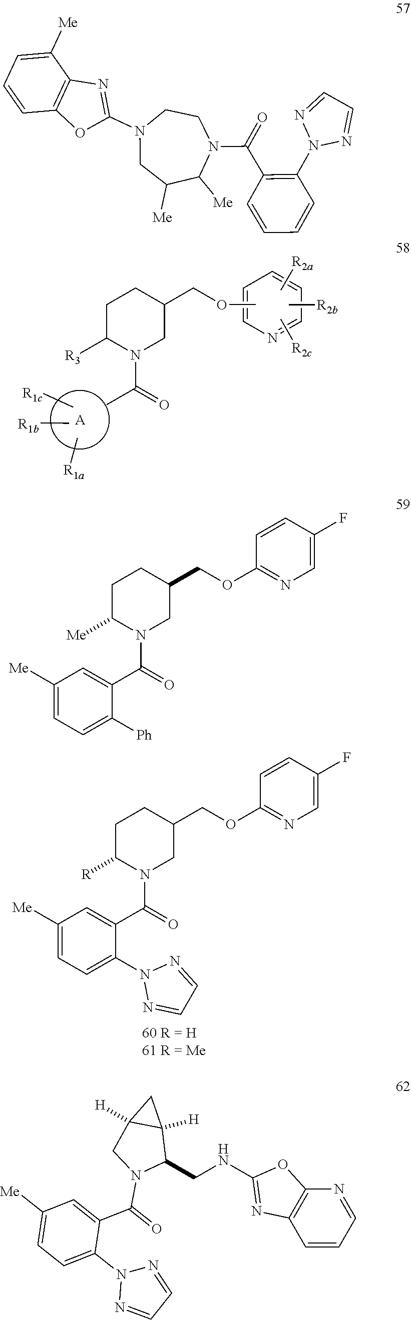

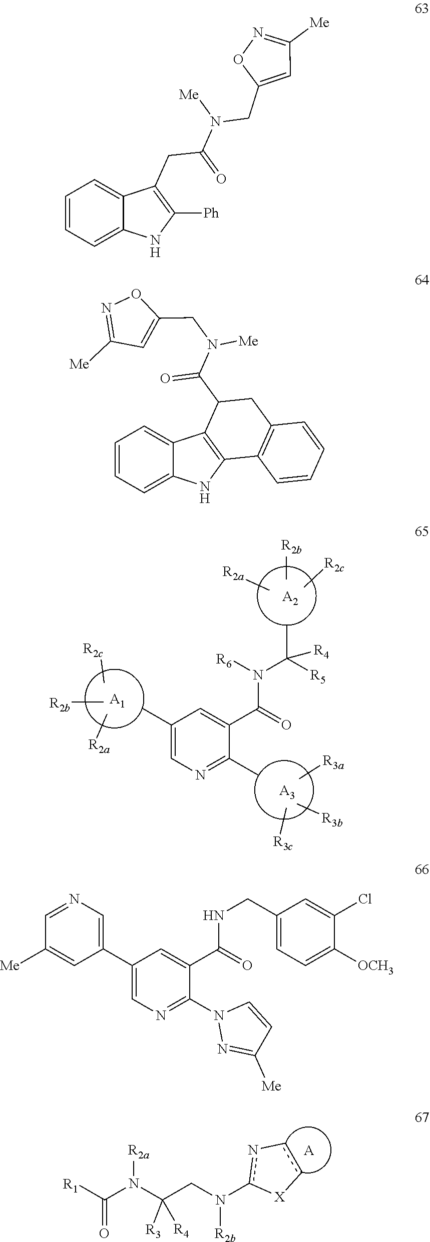

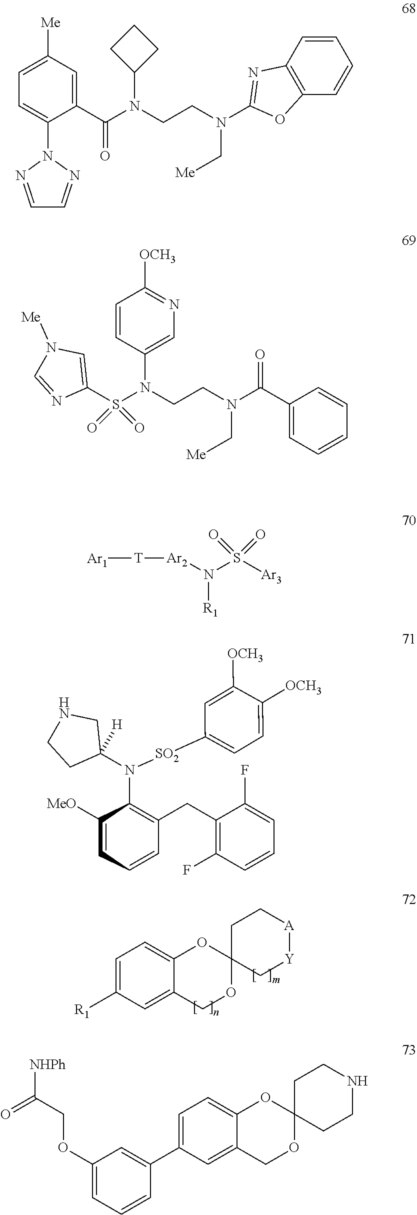

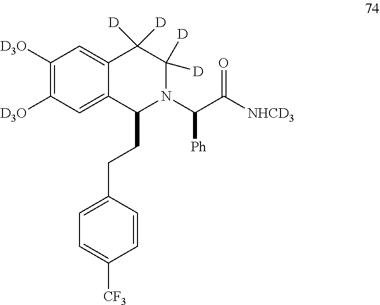

In some embodiments, the OX1R antagonist of the present invention is selected from 2-pyridyloxy-3-substituted-4-nitrile orexin receptor antagonists that are disclosed in WO 2014066196. In some embodiments, the OX1R antagonist of the present invention is selected from the group consisting of:
3-methyl-2-{[(3R,6R)-6-methyl-1-{[2-(2H-1,2,3-triazol-2-1)phenyl]carbonyl}piperidin-3-yl] oxy} pyridine-4-carbonitrile;
2-{[(3R,6R)-1-{[6-methoxy-2-(2H-1,2,3-triazol-2-yl)pyridin-3-yl]carbonyl}-6-methylpiperidin-3-yl]oxy}-3-methylpyridine-4-carbonitrile;
2-{[(3R,6R)-1-{[5-chloro-2-(2H-1,2,3-triazol-2-yl)phenyl]carbonyl}-6-ethylpiperidin-3-yl]oxy}-3-methylpyridine-4-carbonitrile;
2-{[(3R,6R)-1-{[4-chloro-2-(2H-1,2,3-triazol-2-yl)phenyl]carbonyl}-6-ethylpiperidin-3-yl]oxy}-3-methylpyridine-4-carbonitrile;
2-({(3R,6R)-1-[(2-cyclopropyl-6-methoxypyridin-3-yl)carbonyl]-6-methylpiperidin-3-yl}oxy)-3-methylpyridine-4-carbonitrile;
3-methyl-2-{[(3R,6R)-6-methyl-1-{[5-methyl-2-(2H-1,2,3-triazol-2-yl)phenyl]carbonyl}piperidin-3-yl]oxy}pyridine-4-carbonitrile;
2-{[(3R,6R)-1-{[4-methoxy-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[4-methyl-2-(2H-1,2,3-triazol-2-yl)phenyl]carbonyl}piperidin-3-yl]oxy}pyridine-4-carbonitrile;
2-{[(3R,6R)-1-{[4-fluoro-2-(2H-1,2,3-triazol -2-yl)phenyl]carbonyl}-6-methylpiperidin-3-yl]oxy}-3-methylpyridine-4-carbonitrile;
3-methyl-2-{[(3R,6R)-6-methyl-1-{[4-(2H-1,2,3riazol-2-yl)pyridin-3-yl]carbonyl}piperidin-3-yl] oxy} pyridine-4-carbonitrile;
2-{[(3R,6R)-1-{[2-fluoro-6-(2H-1,2,3-triazol-2-yl)phenyl]carbonyl}-6-methylpiperidin-3-yl]oxy}-3-methylpyridine-4-carbonitrile;
2-{[(3R,6R)-1-{[3-fluoro-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[3-methyl-2-(2H-1,2,3-triazol-2-yl)phenyl]carbonyl}piperidin-3-yl]oxy}pyridine-4-carbonitrile;
2-{[(3R,6R)-1-{[2-methoxy-6-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[2-methyl-6-(2H-1,2,3-triazol-2-yl)phenyl]carbonyl}piperidin-3-yl]oxy}pyridine-4-carbonitrile;
2-({(3R,6R)-1-[(2-cyclopropylphenyl)carbonyl]-6-methylpiperidin-3-yl}oxy)-3-methylpyridine-4-carbonitrile;
3-methyl-2-{[(3R,6R)-6-methyl-1-{[2-(trifluoromethyl)phenyl]carbonyl}piperidin-3-yl] oxy} pyridine-4-carbonitrile;
2-{[(3R,6R)-1-{[2-fluoro-6-(1,3-thiazol-2-yl)phenyl]carbonyl}-6-methylpiperidin-3-yl]oxy}-3-methylpyridine-4-carbonitrile;
2-({(3R,6R)-1-[(2-ethoxyphenyl)carbonyl]-6-methylpiperidin-3-yl}oxy)-3-methylpyridine-4-carbonitrile;
3-methyl-2-({(3R,6R)-6-methyl-1-[(4-phenylisothiazol-5-yl)carbonyl]piperidin-3-yl} oxy)pyridine-4-carbonitrile;
3-methyl-2-{[(3R,6R)-6-methyl-1-{[2-(1-methylethoxy)phenyl]carbonyl}piperidin-3-yl]oxy}pyridine-4-carbonitrile;
2-{[(3R,6R)-1-{[2-fluoro-6-(1,3-thiazol-4-yl)phenyl]carbonyl}-6-methylpiperidin-3-yl]oxy}-3-methylpyridine-4-carbonitrile;
3-methyl-2-({(3R,6R)-6-methyl-1-[(3-phenylpyridin-4-yl)carbonyl]piperidin-3-yl} oxy)pyridine-4-carbonitrile;
3-methyl-2-{[(3R,6R)-6-methyl-1-{[3-(1,3-thiazol-4-yl)pyridin-2-yl]carbonyl}piperidin-3-yl] oxy} pyridine-4-carbonitrile;
2-{[(3R,6R)-1-{[2-fluoro-5-(1,3-thiazol-5-yl)pyridin-4-yl]carbonyl}-6-methylpiperidin-3-yl]oxy}-3-methylpyridine-4-carbonitrile;
2-{[(3R,6R)-1-{[5-fluoro-2-(1,3-thiazol-4-yl)phenyl]carbonyl}-6-methylpiperidin-3-yl]oxy}-3methylpyridine-4-carbonitrile;
3-methyl-2-{[(3R,6R)-6-methyl-1-{[2-(1,3-oxazol-2-yl)phenyl]carbonyl}piperidin-3-yl] oxy} pyridine-4-carbonitrile;
2-{[(3R,6R)-1-{[5-fluoro-2-(1,3-thiazol-2-yl)phenyl]carbonyl}-6-methylpiperidin-3-yl]oxy}-3methylpyridine-4-carbonitrile;
3-methyl-2-({(3R,6R)-6-methyl-1-[(2-pyrrolidin-1-ylphenyl)carbonyl]piperidin-3-yl} oxy)pyridine-4-carbonitrile;
3-methyl-2-({(3R,6R)-6-methyl-1-[(2-phenoxyphenyl)carbonyl]piperidin-3-yl}oxy)pyridine-4-carbonitrile;
3-methyl-2-{[(3R,6R)-6-methyl-1-{[2-(1,3-thiazol-4-yl)thiophen-3-yl]carbonyl}piperidin-3-yl] oxy} pyridine-4-carbonitrile;
3-methyl-2-{[(3R,6R)-6-methyl-1-{[2-(1,3-thiazol-2-yl)thiophen-3-yl]carbonyl}piperidin-3-yl]oxy}pyridine-4-carbonitrile;
3-methyl-2-{[(3R,6R)-6-methyl-1-{[2-(1,3-thiazol-4-yl)phenyl]carbonyl}piperidin-3-yl] oxy} pyridine-4-carbonitrile;
2-{[(3R,6R)-1-{[4-fluoro-2-(1,3-thiazol-2-yl)phenyl]carbonyl}-6-methylpiperidin-3-yl]oxy}-3methylpyridine-4-carbonitrile;
2-{[(3R,6R)-1-{[5-fluoro-2-(1,3-thiazol-2-yl)pyridin-3-yl]carbonyl}-6-methylpiperidin-3-yl]oxy}-3-methylpyridine-4-carbonitrile;
3-methyl-2-{[(3R,6R)-6-methyl-1-{[2-(1-methyl-1 H-pyrazol-4-yl)phenyl]carbonyl} piperidin-3 yl] oxy} pyridine-4-carbonitrile;
3-methyl-2-{[(3R,6R)-6-methyl-1-{[3-(1,3-thiazol-2-yl)thiophen-2-yl]carbonyl} piperidin-3-yl] oxy} pyridine-4-carbonitrile;
3-methyl-2-{[(3R,6R)-6-methyl-1-{[3-(1,3-thiazol-4-yl)thiophen-2-yl]carbonyl} piperidin-3-yl] oxy} pyridine-4-carbonitrile;
3-methyl-2-{[(3R,6R)-6-methyl-1-{[2-(2H-1,2,3-triazol-2-yl)thiophen-3-yl]carbonyl}piperidin 3-yl]oxy}pyridine-4-carbonitrile;
3-methyl-2-{[(3R,6R)-6-methyl-1-{[2-(5-methyl-2H-tetrazol-2-yl)phenyl]carbonyl}piperidin-3yl] oxy} pyridine-4-carbonitrile;
2-{[(3R,6R)-1-{[5-(4-fluorophenyl)-2-methyl-1,3-thiazol-4-yl]carbonyl}-6-methylpiperidin-3-yl]oxy}-3-methylpyridine-4-carbonitrile;
3-methyl-2-{[(3R,6R)-6-methyl-1-{[2-(5-methyl-1,2,4-oxadiazol-3-yl)phenyl]carbonyl}piperidin-3-yl]oxy}pyridine-4-carbonitrile;
3-methyl-2-{[(3R,6R)-6-methyl-1-{[2-(4-methyl-1H-pyrazol-1-yl)phenyl]carbonyl}piperidin-3-yl] oxy} pyridine-4-carbonitrile;
3-methyl-2-{[(3R,6R)-6-methyl-1-{[2-(1,3-thiazol-2-yl)phenyl]carbonyl}piperidin-3-yl] oxy} pyridine-4-carbonitrile;
3-methyl-2-{(3R,6R)-6-methyl-1-[(1-methyl-3-phenyl-1H-pyrazol-4-yl)carbonyl]piperidin-3-yl} oxy)pyridine-4-carbonitrile;
3-methyl-2-{[(3R,6R)-6-methyl-1-{[1-methyl-3-(1,3-thiazol-2-yl)-1H-pyrazol-4-yl]carbonyl}piperidin-3-yl]oxy}pyridine-4-carbonitrile;
3-methyl-2-({(3R,6R)-6-methyl-1-[(3-methyl-5-phenylisothiazol-4-yl)carbonyl]piperidin-3-yl} oxy)pyridine-4-carbonitrile;
2-({(3R,6R)-1-[(6-methoxy-2,4′-bipyridin-3-yl)carbonyl]-6-methylpiperidin-3-yl}oxy)-3-methylpyridine-4-carbonitrile;
2-{[(3R,6R)-1-{[2-(6-methoxypyridin-3-yl)phenyl]carbonyl}-6-methylpiperidin-3-yl]oxy}-3-methylpyridine-4-carbonitrile;
3-methyl-2-({(3R,6R)-6-methyl-1-[(4-phenylisothiazol-3-yl)carbonyl]piperidin-3-yl} oxy)pyridine-4-carbonitrile;
2-({(3R,6R)-1-[(2-cyclopropyl-4-methylphenyl)carbonyl]-6-methylpiperidin-3-yl}oxy)-3-methylpyridine-4-carbonitrile;
2-({(3R,6R)-1-[(2-cyclopropyl-4-methoxyphenyl)carbonyl]-6-methylpiperidin-3-yl}oxy)-3-methylpyridine-4-carbonitrile;
2-({(3R,6R)-1-[(2-cyclopropyl-4-fluorophenyl)carbonyl]-6-methylpiperidin-3-yl}oxy)-3-methylpyridine-4-carbonitrile;
2-{[(3R,6R)-1-{[5-(hydroxymethyl)biphenyl-2-yl]carbonyl}-6-methylpiperidin-3-yl]oxy}-3-methylpyridine-4-carbonitrile;
3-methyl-2-{[(3R,6R)-6-methyl-1-{[4-(2H-1,2,3-triazol-2-yl)isothiazol-3-yl]carbonyl}piperidin-3-yl]oxy}pyridine-4-carbonitrile;
2-({(3R,6R)-1-[(5-fluoro-2-pyridin-2-ylphenyl)carbonyl]-6-methylpiperidin-3-yl}oxy)-3-methylpyridine-4-carbonitrile;
3-methyl-2-({(3R,6R)-6-methyl-1-[(2-methyl-5-phenyl-1,3-thiazol-4-yl)carbonyl]piperidin-3-yl} oxy)pyridine-4-carbonitrile;
3-methyl-2-{[(3R,6R)-6-methyl-1-{[2-(3-methyl-1,2,4-oxadiazol-5-yl)phenyl]carbonyl}piperidin-3-yl]oxy}pyridine-4-carbonitrile;
2-({(3R,6R)-1-[(2-ethylphenyl)carbonyl]-6-methylpiperidin-3-yl}oxy)-3-methylpyridine-4-carbonitrile;
3-methyl-2-({(3R,6R)-6-methyl-1-[(2-phenylpyridin-3-yl)carbonyl]piperidin-3-yl} oxy)pyridine-4-carbonitrile;
3-methyl-2-{[(3R,6R)-6-methyl-1-{[2-(methylsulfanyl)phenyl]carbonyl}piperidin-3-yl] oxy} pyridine-4-carbonitrile;
3-methyl-2-{[(3R,6R)-6-methyl-1-{[2-(trifluoromethoxy)phenyl]carbonyl}piperidin-3-yl] oxy} pyridine-4-carbonitrile;
3-methyl-2-({(3R,6R)-6-methyl-1-[(2-pyridin-2-ylcyclopent-1-en-1-yl)carbonyl]piperidin-3-yl} oxy)pyridine-4-carbonitrile;
2-{[(3R,6R)-1-{[4-(fluoromethoxy)-2-(2H-1,2,3-triazol-2-yl)phenyl]carbonyl}-6-methylpiperidin-3-yl]oxy}-3-methylpyridine-4-carbonitrile;
2-{[(3R,6R)-1-{[4-(difluoromethoxy)-2-(2H-1,2,3-triazol-2-yl)phenyl]carbonyl}-6-methylpiperidin-3-yl]oxy}-3-methylpyridine-4-carbonitrile;
2-({(3R,6R)-1-[(2-cyclobutyl-6-methoxypyridin-3-yl)carbonyl]-6-methylpiperidin-3-yl}oxy)-3-methylpyridine-4-carbonitrile;
2-{[(3R,6R)-1-{[6-chloro-4-(2H-1,2,3-triazol-2-yl)pyridin-3-yl]carbonyl}-6-methylpiperidin-3-yl]oxy}-3-methylpyridine-4-carbonitrile;
2-({(3R,6R)-1-[(2-ethoxypyridin-3-yl)carbonyl]-6-methylpiperidin-3-yl}oxy)-3-methylpyridine-4-carbonitrile;
2-{[(3R,6R)-1-{[5-fluoro-2-(2H-1,2,3-triazol-2-yl)phenyl]carbonyl}-6-methylpiperidin-3-yl]oxy}-3-methylpyridine-4-carbonitrile;
3-methyl-2-{[(3R,6R)-6-methyl-1-{[2-(2H-tetrazol-2-yl)phenyl]carbonyl}piperidin-3-yl] oxy} pyridine-4-carbonitrile;
2-{[(3R,6R)-1-{[5-cyano-2-(2H-1,2,3-triazol-2-yl)phenyl]carbonyl}-6-methylpiperidin-3-yl]oxy}-3-methylpyridine-4-carbonitrile;
2-{[(3R,6R)-1-{[6-chloro-2-(2H-1,2,3-triazol-2-yl)pyridin-3-yl]carbonyl}-6-methylpiperidin-3-yl]oxy}-3-methylpyridine-4-carbonitrile;
2-({(3R,6R)-1-[(2,6-dimethoxypyridin-3-yl)carbonyl]-6-methylpiperidin-3-yl}oxy)-3-methylpyridine-4-carbonitrile;
3-methyl-2-{[(3R,6R)-6-methyl-1-{[2-(pyrimidin-2-yl)phenyl]carbonyl}piperidin-3-yl] oxy} pyridine-4-carbonitrile;
3-methyl-2-({(3R,6R)-6-methyl-1-[(3-pyrimidin-2-ylthiophen-2-yl)carbonyl]piperidin-3-yl} oxy)pyridine-4-carbonitrile;
3-methyl-2-({(3R,6R)-6-methyl-1-[(4-methyl-2-yrimidin-2-ylphenyl)carbonyl]piperidin-3-yl} oxy)pyridine-4-carbonitrile;
3-methyl-2-({(3R,6R)-6-methyl-1-[(2-yrimidin-2-ylthiophen-3-yl)carbonyl]piperidin-3-yl} oxy)pyridine-4-carbonitrile;
2-({(3R,6R)-1-[(6-methoxy-2-phenylpyridin-3-yl)carbonyl]-6-methylpiperidin-3-yl}oxy)-3-methylpyridine-4-carbonitrile;
2-{[(3R,6R)-1-{[6-methoxy-2-(1-methyl-1H-pyrazol-5-yl)pyridin-3-yl]carbonyl}-6-methylpiperidin-3-yl]oxy}-3-methylpyridine-4-carbonitrile;
2-{[(3R,6R)-1-{[6-methoxy-2-(1-methyl-1H-pyrazol-4-yl)pyridin-3-yl]carbonyl}-6-methylpiperidin-3-yl]oxy}-3-methylpyridine-4-carbonitrile;
2-{[(3R,6R)-1-{[6-methoxy-2-(1-methyl-1H-pyrazol-3-yl)pyridin-3-yl]carbonyl}-6-methylpiperidin-3-yl]oxy}-3-methylpyridine-4-carbonitrile;
2-({(3R,6R)-1-[(6-methoxy-2,3′-bipyridin-3-yl)carbonyl]-6-methylpiperidin-3-yl}oxy)-3-methylpyridine-4-carbonitrile;
2-({(3R,6R)-1-[(6-methoxy-2,2′-bipyridin-3-yl)carbonyl]-6-methylpiperidin-3-yl}oxy)-3-methylpyridine-4-carbonitrile;
2-{[(3R,6R)-1-{[6-Methoxy-2-(methylsulfanyl)pyridin-3-yl]carbonyl}-6-methylpiperidin-3-yl]oxy}-3-methylpyridine-4-carbonitrile;
2-{[(3R,6R)-1-{[6-methoxy-4-(2H-1,2,3 riazol-2-yl)pyridin-3-yl]carbonyl}-6-methylpiperidin
3-yl]oxy}-3-methylpyridine-4-carbonitrile;
3-methyl-2-{[(3R,6R)-6-methyl -{[6-(methylsulfanyl)-2-(2H-1,2,3-triazol-2-yl)pyridin-3-yl]carbonyl}piperidin-3-yl]oxy}pyridine-4-carbonitrile;
2-{[(3R,6R)-1-{[2-(dimethylamino)-6-methoxypyridin-3-yl]carbonyl}-6-methylpiperidin-3-yl]oxy}-3-methylpyridine-4-carbonitrile;
2-{[(3R,6R)-1-{[6-(fluoromethoxy)-2-(2H-1,2,3-triazol-2-yl)pyridin-3-yl]carbonyl}-6-methylpiperidin-3-yl]oxy}-3-methylpyridine-4-carbonitrile;
2-{[(3R,6R)-1-{[6-bromo-2-(2H-1,2,3-triazol-2-yl)pyridin-3-yl]carbonyl}-6-methylpiperidin-3 yl]oxy}-3-methylpyridine-4-carbonitrile;
2-{[(3R,6R)-1-{[6-ethenyl-2-(2H-1,2,3 riazol-2-yl)pyridin-3-yl]carbonyl}-6-methylpiperidin-yl]oxy}-3-methylpyridine-4-carbonitrile;
3-chloro-2-{[(3R,6R)-6-methyl-1-{[2-(2H-1,2,3-triazol-2-yl)phenyl]carbonyl} piperidin-3-yl] oxy} pyridine-4-carbonitrile;
3-cyclopropyl-2-{[(3R,6R)-6-methyl-1-{[2-(2H-1,2,3-triazol-2-yl)phenyl]carbonyl}piperidin-3 yl] oxy} pyridine-4-carbonitrile;
3-ethyl-2-{[(3R,6R)-6-methyl-1-{[2-(2H-1,2,3-triazol-2-yl)phenyl]carbonyl}piperidin-3-yl] oxy} pyridine-4-carbonitrile;
2-{[(3R,6R)-6-methyl-1-{[2-(2H-1,2,3-triazol-2-yl)phenyl]carbonyl}piperidin-3-yl]oxy}pyridine-3,4-dicarbonitrile;
3-(methylsulfanyl)-2-{[(3R,6R)-6-methyl-1-{[2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[2-(2H-1,2,3-triazol-2-yl)phenyl]carbonyl} piperidin-3-yl] oxy} pyridine-4-carbonitrile;
2-{[(3R,6R)-1-{[3-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[4-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[5-fluoro-2-(2H-1,2,3-triazol-2-yl)phenyl]carbonyl}-6-methylpiperidin-3-yl]oxy}-3-methoxypyridine-4-carbonitrile;
2-{[(3R,6R)-1-{[2-fluoro-6-(2H-1,2,3-triazol-2-yl)phenyl]carbonyl}-6-methylpiperidin-3-yl]oxy}-3-methoxypyridine-4-carbonitrile;
3-methoxy-2-{[(3R,6R)-6-methyl-1-{[3-methyl-2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[4-methyl-2-(2H-1,2,3-triazol-2-yl)phenyl]carbonyl}piperidin-3-yl]oxy}pyridine-4-carbonitrile;
3-methoxy-2-{[(3R,6R)-6-methyl-1-{[5-methyl-2-(2H-1,2,3-triazol-2-yl)phenyl]carbonyl}piperidin-3-yl]oxy}pyridine-4-carbonitrile;
3-methoxy-2-{[(3R,6R)-1-{[6-methoxy-2-(2H-1,2,3-triazol-2-yl)pyridin-3-yl]carbonyl}-6-methylpiperidin-3-yl]oxy}pyridine-4-carbonitrile;
2-({(3R,6R)-1-[(2-cyclobutylphenyl)carbonyl]-6-methylpiperidin-3-yl}oxy)-3-methoxypyridine-4-carbonitrile;
3-methoxy-2-{[(3R,6R)-6-methyl-1-{[2-(2H-1,2,3-triazol-2-yl)thiophen-3-yl]carbonyl}piperidin-3-yl]oxy}pyridine-4-carbonitrile;
3-methoxy-2-({(3R,6R)-6-methyl-1-[(2-pyrimidin-2-ylphenyl)carbonyl]piperidin-3-yl} oxy)pyridine-4-carbonitrile;
3-methoxy-2-({(3R,6R)-6-methyl-1-[(2-pyrimidin-2-ylthiophen-3-yl)carbonyl]piperidin-3-yl} oxy)pyridine-4-carbonitrile;
methyl 4-cyano-2-{[(3R,6R)-1-{[3-fluoro-2-(pyrimidin-2-yl)phenyl]carbonyl}-6-methylpiperidin-3-yl]oxy}pyridine-3-carboxylate
2-({(3R,6R)-1-[(4-fluoro-2̂yrimidin-2-ylphenyl)carbonyl]-6-methylpiperidin-3-yl}oxy)-3-methoxypyridine-4-carbonitrile;
2-({(3R,6R)-1-[(5-fluoro-2̂yrimidin-2-ylphenyl)carbonyl]-6-methylpiperidin-3-yl}oxy)-3-methoxypyridine-4-carbonitrile;
2-({(3R,6R)-1-[(2-fluoro-6̂yrimidin-2-ylphenyl)carbonyl]-6-methylpiperidin-3-yl}oxy)-3-methoxypyridine-4-carbonitrile;
3-methoxy-2-({(3R,6R)-1-[(6-methoxy-2-pyrimidin-2-ylpyridin-3-yl)carbonyl]-6-methylpiperidin-3-yl}oxy)pyridine-4-carbonitrile;
3-methyl-2-{[(3R,6R)-6-methyl-1-{[2-(1-methylethoxy)pyridin-3-yl]carbonyl}piperidin-3-yl]oxy}pyridine-4-carbonitrile; and
3-methoxy-2-{[(3R,6R)-6-methyl-1-{[2-(2H-tetrazol-2-yl)phenyl]carbonyl}piperidin-3-yl] oxy} pyridine-4-carbonitrile;
and pharmaceutically acceptable salts thereof.

In some embodiments, the OX1R antagonist of the present invention is selected from 2-pyridylamino-4-nitrile-piperidinyl orexin receptor antagonists that are disclosed in WO 2014085208 A1. In some embodiments, the OX1R antagonist of the present invention is selected from the group consisting of:
2-(((3R,6R)-1-(2-(2H-1,2,3-triazol-2-yl)benzoyl)-6-methylpiperidin-3-yl)amino)isonicotinonitrile;
3-methoxy-2-{[(3R,6R)-6-methyl-1-{[2-(2H-1,2,3-triazol-2-yl)phenyl]carbonyl} piperidin-3-yl] amino} pyridine-4-carbonitrile;
2-(((3R,6R)-1-(2-(2H etrazol-2-yl)benzoyl)-6-methylpiperidin-3-yl)amino)isonicotinonitrile;
2-(((3R,6R)-1-(2-(2H-1,2,3-triazol-2-yl)benzoyl)-6-methylpiperidin-3-yl)amino)-3-methylisonicotinonitrile;
3-methyl-2-{[(3R,6R)-6-methyl-1-{[2-(2H-tetrazol-2-yl)phenyl]carbonyl}piperidin-3-yl] amino} pyridine-4-carbonitrile;
2-((3R,6R)-1-(2-(2H-1,2,3-triazol-2-yl)thiophene-3-carbonyl)-6-methylpiperidin-3-ylamino)-3methoxyisonicotinonitrile;
3-methoxy-2-((3R,6R)-6-methyl-1-(2-(yrimidin-2-yl)thiophene-3-carbonyl)piperidin-3-ylamino)isonicotinonitrile;
2-(((3R,6R)-1-(2-(2H-1,2,3-triazol-2-yl)benzoyl)-6-methylpiperidin-3-yl)(methyl)amino)isonicotinonitrile;
2-{methyl[(3R,6R)-6-methyl-1-{[2-(2H-tetrazol-2-yl)phenyl]carbonyl}piperidin-3-yl] amino} pyridine-4-carbonitrile;
3-methoxy-2-{methyl[(3R,6R)-6-methyl-1-{[2-(2H-1,2,3-triazol-2-yl)thiophen-3-yl]carbonyl}piperidin-3-yl]amino}pyridine-4-carbonitrile;
3-methoxy-2-(methyl{(3R,6R)-6-methyl-1-[(2-pyrimidin-2-ylthiophen-3-yl)carbonyl]piperidin3-yl}amino)pyridine-4-carbonitrile;
2-{ethyl[(3R,6R)-6-methyl-1-{[2-(2H-1,2,3-triazol-2-yl)phenyl]carbonyl}piperidin-3-yl] amino} pyridine-4-carbonitrile;
2-{[(3R,6R)-6-methyl-1-{[2-(2H-1,2,3-triazol-2-yl)phenyl]carbonyl}piperidin-3-yl](prop-2-en-1-yl)amino} pyridine-4-carbonitrile;
2-{[(3R,6R)-6-methyl-1-{[2-(2H-1,2,3-triazol-2-yl)phenyl]carbonyl}piperidin-3-yl] (propyl)amino} pyridine-4-carbonitrile;
N-((3R,6R)-1-(2-(2H-1,2,3-triazol-2-yl)benzoyl)-6-methylpiperidin-3-yl)-N-(4-cyanopyridin-2 yl)acetamide;
methyl 4-cyano-2-{[(3R,6R)-6-methyl-1-{[2-(2H-1,2,3-triazol-2-yl)phenyl]carbonyl}piperidine-3-yl] amino} pyridine-3-carboxylate;
methyl 4-cyano-2-({(3R,6R)-6-methyl-1-[(2-pyrimidin-2-ylphenyl)carbonyl]piperidin-3-yl}amino)pyridine-3-carboxylate;
and pharmaceutically acceptable salts thereof.

In some embodiments, the OX1R antagonist of the present invention is selected from 2-pyridyloxy-4-nitrile orexin receptor antagonists that are disclosed in WO 2013059222 A1. In some embodiments, the OX1R antagonist of the present invention is selected from the group consisting of
2-{[(3R,6R)-6-methyl-1-{[2-(2H-1,2,3-triazol-2-yl)phenyl]carbonyl}piperidin-3-yl]oxy}pyridine-4-carbonitrile;
2-({(3R,6R)-6-methyl-1-[(2-pyrimidin-2-ylphenyl)carbonyl]piperidin-3-yl}oxy)pyridine-4-carbonitrile;
2-{[(3R,6R)-6-methyl-1-{[2-(5-methyl-1,2,4-oxadiazol-3-yl)phenyl]carbonyl}piperidin-3-yl]oxy}pyridine-4-carbonitrile;
2-{[(3R,6R)-6-methyl-1-{[2-methyl-6-(2H-1,2,3-triazol-2-yl)phenyl]carbonyl}piperidin-3-yl]oxy}pyridine-4-carbonitrile;
2-{[(3R,6R)-6-methyl-1-{[3-methyl-2-(2H-1,2,3-triazol-2-yl)phenyl]carbonyl}piperidin-3-yl]oxy}pyridine-4-carbonitrile;
2-{[(3R,6R)-1-{[3-fluoro-2-(2H-1,2,3-triazol-2-yl)phenyl]carbonyl}-6-methylpiperidin-3-yl]oxy}pyridine-4-carbonitrile;
2-{[(3R,6R)-1-{[4-chloro-2-(2H-1,2,3-triazol-2-yl)phenyl]carbonyl}-6-methylpiperidin-3-yl]oxy}pyridine-4-carbonitrile;
2-{[(3R,6R)-1-{[4-fluoro-2-(2H-1,2,3-triazol-2-yl)phenyl]carbonyl}-6-methylpiperidin-3-yl]oxy}pyridine-4-carbonitrile;
2-{[(3R,6R)-6-methyl-1-{[4-methyl-2-(2H-1,2,3-triazol-2-yl)phenyl]carbonyl}piperidin-3-yl]oxy}pyridine-4-carbonitrile;
2-{[(3R,6R)-6-methyl-1-{[2-(2H-1,2,3-triazol-2-yl)thiophen-3-yl]carbonyl}piperidin-3-yl]oxy}pyridine-4-carbonitrile;
2-{[(3R,6R)-6-methyl-1-{[4-(2H-1,2,3-triazol-2-yl)isothiazol-3-yl]carbonyl}piperidin-3-yl]oxy}pyridine-4-carbonitrile;
2-{[(3R,6R)-6-methyl-1-{[1-methyl-3-(2H-1,2,3-triazol-2-yl)-1 H-pyrazol-4-yl]carbonyl}piperidin-3-yl]oxy}pyridine-4-carbonitrile;
2-{[(3R,6R)-6-methyl-1-{[3-methyl-5-(2H-1,2,3-triazol-2-yl)isothiazol-4-yl]carbonyl}piperidin-3-yl]oxy} pyridine -4-carbonitrile;
2-{[(3R,6R)-6-methyl-1-{[3-(2H-1,2,3-triazol-2-yl)thiophen-2-yl]carbonyl}piperidin-3-yl]oxy}pyridine-4-carbonitrile;
2-{[(3R,6R)-1-{[5-bromo-2-(2H-1,2,3-triazol-2-yl)phenyl]carbonyl}-6-methylpiperidin-3-yl]oxy}pyridine-4-carbonitrile;
2-{[(3R,6R)-1-{[5-chloro-2-(2H-1,2,3-triazol-2-yl)phenyl]carbonyl}-6-methylpiperidin-3-yl]oxy}pyridine-4-carbonitrile;
2-{[(3R,6R)-1-{[6-methoxy-2-(2H-1,2,3-triazol-2-yl)pyridin-3-yl]carbonyl}-6-methylpiperidin-3-yl]oxy} pyridine -4-carbonitrile;
3-({(2R,5R)-5-[(4-cyanopyridin-2-yl)oxy]-2-methylpiperidin-1-yl}carbonyl)-4-(2H-1,2,3-triazol-2-yl)benzamide;
2-{[(3R,6R)-1-{[4-cyano-2-(2H-1,2,3-triazol-2-yl)phenyl]carbonyl}-6-methylpiperidin-3-yl]oxy}pyridine-4-carbonitrile;
2-({(3R,6R)-1-[(2-cyclopropyl-6-methoxypyridin-3-yl)carbonyl]-6-methylpiperidin-3-yl} oxy)pyridine-4-carbonitrile;
2-{[(3R,6R)-1-{[4-ethoxy-2-(2H-1,2,3-triazol-2-yl)phenyl]carbonyl}-6-methylpiperidin-3-yl]oxy}pyridine-4-carbonitrile;
2-{[(3R,6R)-1-{[2-fluoro-6-(2H-1,2,3-triazol-2-yl)phenyl]carbonyl}-6-methylpiperidin-3-yl]oxy}pyridine-4-carbonitrile;
2-{[(3R,6R)-1-{[4-(fluoromethoxy)-2-(2H-1,2,3-triazol-2-yl)phenyl]carbonyl}-6-methylpiperidin-3-yl]oxy}pyridine-4-carbonitrile;
2-{[(3R,6R)-1-{[4-(difluoromethoxy)-2-(2H-1,2,3-triazol-2-yl)phenyl]carbonyl}-6-methylpiperidin-3-yl]oxy}pyridine-4-carbonitrile;
2-{[(3R,6R)-1-{[4-(2-hydroxyethoxy)-2-(2H-1,2,3-triazol-2-yl)phenyl]carbonyl}-6-methylpiperidin-3-yl]oxy}pyridine-4-carbonitrile;
2-{[(3R,6R)-6-methyl-1-{[6-methyl-2-(2H-1,2,3-triazol-2-yl)pyridin-3-yl]carbonyl}piperidin-3 yl]oxy}pyridine-4-carbonitrile;
2-{[(3R,6R)-6-methyl-1-{[2-(2H-1,2,3-triazol-2-yl)-6-(trifluoromethyl)pyridin-3-yl]carbonyl}piperidin-3-yl]oxy}pyridine-4-carbonitrile;
2-({(3R,6R)-1-[(2-chloro-6-methoxypyridin-3-yl)carbonyl]-6-methylpiperidin-3-yl} oxy)pyridine-4-carbonitrile;
2-{[(3R,6R)-1-{[4-chloro-2-(2H-1,2,3-triazol-2-yl)phenyl]carbonyl}-6-methylpiperidin-3-yl]oxy}pyridine-4-carbonitrile;
2-{[(3R,6R)-6-methyl-1-{[2-(2H-tetrazol-2-yl)phenyl]carbonyl}piperidin-3-yl]oxy}pyridine-4-carbonitrile;
2-({(3R,6R)-6-methyl-1-[(3-pyrimidin-2-ylthiophen-2-yl)carbonyl]piperidin-3-yl}oxy)pyridine-4-carbonitrile;
2-({(3R,6R)-6-methyl-1-[(2-pyrimidin-2-ylthiophen-3-yl)carbonyl]piperidin-3-yl}oxy)pyridine-4-carbonitrile;
2-({(3R,6R)-1-[(3-fluoro-2-pyrimidin-2-ylphenyl)carbonyl]-6-methylpiperidin-3-yl}oxy)pyridine-4-carbonitrile;
2-({(3R,6R)-1-[(4-fluoro-2-pyrimidin-2-ylphenyl)carbonyl]-6-methylpiperidin-3-yl}oxy)pyridine-4-carbonitrile;
2-({(3R,6R)-1-[(5-fluoro-2-pyrimidin-2-ylphenyl)carbonyl]-6-methylpiperidin-3-yl}oxy)pyridine-4-carbonitrile; and
2-({(3R,6R)-1-[(2-fluoro-6-pyrimidin-2-ylphenyl)carbonyl]-6-methylpiperidin-3-yl} oxy)pyridine-4-carbonitrile;
and pharmaceutically acceptable salts thereof.

In some embodiments, the OX1R antagonist of the present invention is selected from 2-pyridyloxy-4-ester orexin receptor antagonists that are disclosed in WO 2014099696 A1. In some embodiments, the OX1R antagonist of the present invention is selected from the group consisting of:
methyl 2-{[(3R,6R)-6-methyl-1-{[2-(2H-1,2,3-triazol-2-yl)phenyl]carbonyl}piperidin-3-yl]oxy}pyridine-4-carboxylate;
2-{[(3R,6R)-6-methyl-1-{[2-(2H-1,2,3-triazol-2-yl)phenyl]carbonyl}piperidin-3-yl] oxy} pyridine-4-carboxylic acid;
methyl 2-{[(3R,6R)-6-methyl-1-{[2-(2H-1,2,3-triazol-2-yl)phenyl]carbonyl}piperidin-3-yl]oxy}-5-(trifluoromethyl)pyridine-4-carboxylate;
methyl 5-bromo-2-{[(3R,6R)-6-methyl-1-{[2-(2H-1,2,3-triazol-2-yl)phenyl]carbonyl}piperidin-3-yl]oxy}pyridine-4-carboxylate;
methyl 3-chloro-2-{[(3R,6R)-6-methyl-1-{[2-(2H-1,2,3-triazol-2-yl)phenyl]carbonyl}piperidin-3-yl]oxy}pyridine-4-carboxylate;
dimethyl 2-{[(3R,6R)-6-methyl-1-{[2-(2H-1,2,3-triazol-2-yl)phenyl]carbonyl}piperidin-3-yl]oxy}pyridine-3,4-dicarboxylate;
methyl 2-methyl-6-{[(3R,6R)-6-methyl-1-{[2-(2H-1,2,3-triazol-2-yl)phenyl]carbonyl}piperidin-3-yl]oxy}pyridine-4-carboxylate;
methyl 3-fluoro-2-{[(3R,6R)-6-methyl-1-{[2-(2H-1,2,3-triazol-2-yl)phenyl]carbonyl}piperidin-3-yl]oxy}pyridine-4-carboxylate;
methyl 2-{[(3R,6R)-1-{[6-methoxy-2-(2H-1,2,3-triazol-2-yl)pyridin-3-yl]carbonyl}-6-methylpiperidin-3-yl]oxy}pyridine-4-carboxylate;
methyl 5-methoxy-2-{[(3R,6R)-6-methyl-1-{[2-(2H-1,2,3-triazol-2-yl)phenyl]carbonyl}piperidin-3-yl]oxy}pyridine-4-carboxylate;
methyl 5-methyl-2-{[(3R,6R)-6-methyl-1-{[2-(2H-1,2,3-triazol-2-yl)phenyl]carbonyl}piperidin-3-yl]oxy}pyridine-4-carboxylate;
methyl 2-({(3R,6R)-1-[(6-methoxy-2-pyrimidin-2-ylpyridin-3-yl)carbonyl]-6-methylpiperidin-3-yl}oxy)pyridine-4-carboxylate;
and pharmaceutically acceptable salts thereof.

In some embodiments, the OX1R antagonist of the present invention is selected from tertiary amide orexin receptor antagonists that are disclosed in WO 2011053522 A1. In some embodiments, the OX1R antagonist of the present invention are selected from the group consisting of:
N-[2-(5,6-dimethoxy-3˜pyridinyl)ethyl]-N-[3-(4-methoxyphenyl)-1-methylpropyl]-6-methyl-2-pyridinecarboxamide;
6-chloro-N-[2-(5,6-dimethoxypyridinecarboxamide;
N-f2-(5ₛ6-dimethoxy-2-pyridinyl)ethyl]-6-methyl-N-(1-methyl-3-phenylpropyl)-2-pyridinecarboxamide;
N-[2-(5,6-dimethoxypyridin-2-yl)ethyl]-6-(dimethylamino)-N-[4-(4-methoxyphenyl)butan-2-yl]pyridine-2-carboxamide;
6-chloro-N-[2-(5,6-dimethoxypyridin-2-yl)ethyl]-N-(4-phenylbutan-2-yl)pyridine-2-carboxamide
N-[2-(5_{}},6-dimethoxypyridin-2-yl)ethyl]-3-methyl-N-(4-phenylbutan-2˜yl)benzamide;
6-bromo-N-[2-(5,6-dimethoxypyridin-2-yl)ethyl]-N-(4-phenylbutan-2-yl)pyridine-2-carboxamide;
N-[2-(5,6-dimethoxypyridin-2-yl)ethyl]-6-fluoro-N-(4-phenylbutan-2-yl)pyridine-2-carboxamide;
N-[2-(5,6-dimethoxypyridin-2-yl)ethyl]-N-(4-phenylbutan-2-yl)-6-(propan-2-yl)pyridine-2-carboxamide;
6-cyano-N-[2-(5,6-dimethoxypyridin-2-yl)ethyl]-N-(4-phenylbutan-2-yl)pyridine-2-carboxamide
6-cyclopropyl-N-[2-(5,6-dimethoxypyridin-2-yl)ethyl]-N-(4-phenylbutan-2-yl)pyridine-2-carboxamide;
N-[2-(5,6-dimethoxypyridin-2-yl)ethyl]˜6-ethyl-N-(4-phenylbutan-2-yl)pyridine-2-carboxamide
N-[2-(5,6-dimethoxypyridin-2-yl)ethyl]-6-methylcarboxamide;
6-chloro-N-[2-(5,6-dimethoxypyridin-2-yl)methylcarboxamide;
and pharmaceutically acceptable salts thereof.

In some embodiments, the OX1R antagonist of the present invention is selected from 3-ester-4-substituted orexin receptor antagonists that are disclosed in WO 2014099697 A1. In some embodiments, the OX1R antagonist of the present invention is selected from the group consisting of:
ethyl 4-methyl-2-{[(3R,6R)-6-methyl-1-{[2-(2H-1,2,3-triazol-2-yl)phenyl]carbonyl}piperidin-3-yl] oxy} pyridine-3-carboxylate;
methyl 4-methyl-2-{[(3R,6R)-6-methyl-1-{[2-(2H-1,2,3-triazol-2-yl)phenyl]carbonyl}piperidin3-yl]oxy}pyridine-3-carboxylate;
methyl 4-(methylsulfanyl)-2-{[(3R,6R)-6-methyl-1-{[2-(2H-1,2,3-triazol-2-yl)phenyl]carbonyl}piperidin-3-yl]oxy}pyridine-3-carboxylate;
methyl 2-({(3R,6R)-6-methyl-1-[(2-pyrimidin-2-ylphenyl)carbonyl]piperidin-3-yl}oxy)-4-(methylsulfanyl)pyridine-3-carboxylate;
methyl 2-({(3R,6R)-1-[(5-fluoro-2-pyrimidin-2-ylphenyl)carbonyl]-6-methylpiperidin-3-yl}oxy)-4-(methylsulfanyl)pyridine-3-carboxylate;
methyl 2-({(3R,6R)-1-[(4-fluoro-2-pyrimidin-2-ylphenyl)carbonyl]-6-methylpiperidin-3-yl}oxy)-4-(methylsulfanyl)pyridine-3-carboxylate;
methyl 2-({(3R,6R)-1-[(3-fluoro-2-pyrimidin-2-ylphenyl)carbonyl]-6-methylpiperidin-3-yl}oxy)-4-(methylsulfanyl)pyridine-3-carboxylate;
methyl 4-azetidin-1-yl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-(4-methylpiperazin-1-yl)-2-{[(3R,6R)-6-methyl-1-{[2-(2H-1,2,3-triazol-2-yl)phenyl]carbonyl}-piperidin-3-yl]oxy}pyridine-3-carboxylate;
methyl 4-ethyl-2-{[(3R,6R)-6-methyl-1-{[2-(2H-1,2,3-triazol-2-yl)phenyl]carbonyl}piperidin-3 yl] oxy} pyridine-3-carboxylate;
methyl 4-tert-butyl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-(1-methylethyl)-2-{[(3R,6R)-6-methyl-1-{[2-(2H-1,2,3-triazol-2-yl)phenyl]carbonyl}piperidin-3-yl]oxy}pyridine-3-carboxylate;
methyl 4-cyclopropyl-2-{[(3R,6R)-6-methyl-1-{[2-(2H-1,2,3-triazol-2-yl)phenyl]carbonyl}piperidin-3-yl]oxy}pyridine-3-carboxylate;
methyl 4-cyclobutyl-2-{[(3R,6R)-6-methyl-1-{[2-(2H-1,2,3-triazol-2-yl)phenyl]carbonyl}piperidin-3-yl]oxy}pyridine-3-carboxylate;
methyl 2-methyl-6-{[(3R,6R)-6-methyl-1-{[2-(2H-1,2,3-triazol-2-yl)phenyl]carbonyl}piperidin3-yl]oxy}benzoate;
methyl 2-(1-methylethyl)-6-{[(3R,6R)-6-methyl-1-{[2-(2H-1,2,3-triazol-2-yl)phenyl]carbonyl}piperidin-3-yl] oxy} benzoate; and
ethyl 4-ethyl-2-{[(3R,6R)-6-methyl-1-{[2-(2H-1,2,3-triazol-2-yl)phenyl]carbonyl}piperidin-3-yl] oxy} pyridine-3-carboxylate;
and pharmaceutically acceptable salts thereof.

In some embodiments, the OX1R antagonist of the present invention is selected from the group consisting of 2,5-disubstituted thiomorpholine orexin receptor antagonists that are disclosed in WO 2013059163 A1. In some embodiments, the OX1R antagonist of the present invention is selected from the group consisting of:
[(2R,5R)-2-{[(5-fluoropyridin-2-yl)oxy]methyl}-5-methylthiomorpholin-4-yl][5-methyl-2-(2H-1,2,3-triazol-2-yl)phenyl]methanone;
(2R,5R)-5-methyl-2-[(pyridin-2-yloxy)methyl]-4-{[2-(2H-1,2,3-triazol-2-yl)phenyl] carbonyl} thiomorpholine;
(2R,5R)-2-{[(5-fluoropyridin-2-yl)oxy]methyl}-5-methyl-4-{[2-(2H-1,2,3-triazol-2-yl)phenyl] carbonyl} thiomorpholine;
(2R,5R)-5-methyl-2-{[(5-methylpyridin-2-yl)oxy]methyl}-4-{[2-(2H-1,2,3-triazol-2-yl)phenyl] carbonyl} thiomorpholine;
(2R,5R)-2-{[(5-chloropyridin-2-yl)oxy]methyl}-5-methyl-4-{[2-(2H-1,2,3-triazol-2-yl)phenyl] carbonyl} thiomorpholine;
6-{[(2R,5R)-5-methyl-4-{[2-(2H-1,2,3-triazol-2-yl)phenyl]carbonyl}thiomorpholin-2-yljmethoxy} pyridine-3-carbonitrile;
(2R,5R)-5-methyl-4-{[2-(2H-1,2,3-triazol-2-yl)phenyl]carbonyl}-2-({[5-(trifluoromethyl)pyridin-2-yl]oxy}methyl)thiomorpholine;
(2R,5R)-2-{[(3-chloropyridin-2-yl)oxy]methyl}-5-methyl-4-{[2-(2H-1,2,3-triazol-2-yl)phenyl] carbonyl} thiomorpholine;
2-{[(2R,5R)-5-methyl-4-{[2-(2H-1,2,3-triazol-2-yl)phenyl] carbonyl}thiomorpholin-2-yljmethoxy} pyridine-3-carbonitrile;
(2R,5R)-5-methyl-2-{[(4-methylpyridin-2-yl)oxy]methyl}-4-{[2-(2H-1,2,3-triazol-2-yl)phenyl] carbonyl} thiomorpholine;
(2R,5R)-5-methyl-2-[(pyrimidin-2-yloxy)methyl]-4-{[2-(2H-1,2,3-triazol-2-yl)phenyl] carbonyl} thiomorpholine;
(2R,5R)-2-{[(5-chloro-4-methylpyrimidin-2-yl)oxy]methyl}-5-methyl-4-{[2-(2H-1,2,3-triazol-2-yl)phenyl] carbonyl} thiomorpholine;
(2R,5R)-2-{[(4-chloro-5-methylpyrimidin-2-yl)oxy]methyl}-5-methyl-4-{[2-(2H,2,3-M yl)phenyl] carbonyl} thiomorpholine;
(2R,5R)-2-{[(4-chloro-5-methoxypyrimidin-2-yl)oxy]methyl}-5-methyl-4-{[2-(2H-1,22-yl)phenyl]carbonyl}thiomorpholine;
[(2R,5R)-2-{[(5-fluoropyridin-2-yl)oxy]methyl}-5-methyl-1-oxidothiomorpholin-4-1,2,3-triazol-2-yl)phenyl]methanone;
[(2R,5R)-2-{[(5-fluoropyridin-2-yl)oxy]methyl}-5-m(2H-1,2,3-triazol-2-yl)phenyl]methanone; and
{(2R,5R)-5-methyl-2-[(pyridine-2-ylsulfanyl)methyl]thiomorpholin-4-yl}[2-(2H-1,2,3-triazol-2-yl)phenyl]methanone;
and pharmaceutically acceptable salt thereof.

In some embodiments, the OX1R antagonist of the present invention is selected from piperidinyl alkyne orexin receptor antagonists that are disclosed in WO 2013062857 A1. In some embodiments, the OX1R antagonist of the present invention is selected from the group consisting of:
[(2R,5R)-2-methyl-5-(yridin-2-ylemynyl)piperidin-1-yl][2-(2H-1,2,3-triyl)phenyl]methanone;
2-{[(3S,6R)-6-methyl-1-{[2-(2H-1,2,3-triazol-2-yl)phenyl]carbonyl}piperidin-3-yl] ethynyl} pyridine;
4-{[(3S,6R)-6-methyl-1-{[2-(2H,2₅3-tria2ol-2-yl)phenyl]carbonyl}piperidin-3-yl] ethynyl} pyridine;
3-{[(3S,6R)-6-methyl-1-{[2-(2H-1,2,3-triazol-2-yl)phenyl] carbonyl} piperidin-3-yl] ethynyl} pyridine;
3-{[(3S,6R)-6-methyl-1-{[2-(2H-1,2,3-triazol-2-yl)phenyl]carbonyl}piperidin-3-yl]ethynyl}pyridin-2-ol;
3-{[(3S,6R)-6-methyl-1-{[2-(2H-1,2,3-triazol-2-yl)phenyl]carbonyl}piperidin-3-yl]ethynyl}pyridin-4-ol;
(5-{[(3S,6R)-6-methyl-1-{[2-(2H-1,2,3-triayl]ethynyl}pyridin-2-yl)methanol;
(6-{[(3S,6R)-6-methyl-1-{[2-(2H-1,2,3-triazol-2-yl)phenyl]carbonyl}piperidin-3-yl]ethynyl}pyridin-3-yl)methanol;
(6-{[(3S,6R)-6-methyl-1-{[2-(2H-1,2,3-triazol-2-yl)phenyl]carbonyl}piperidin-3-yl]ethynyl}pyridin-2-yl)methanol;
(2-{[(3S,6R)-6-methyl-1-{[2-(2H,2,3-triazol-2-yl)phenyl]carbonyl}piperidin-3-yl]ethynyl}pyridin-4-yl)methanol;
(4-{[(3S,6R)-6-methyl-1-{[2-(2H-1,2_{J}3-triazol-2-yl)phenyl]carbonyl}piperidin-4-yl] ethynyl} pyridin-2-yl)methanol;
{(2R,5S)-5-[(5-fluoropyridin-2-yl)ethynyl]-2-methylpiperidin-1-yl}[5-methyl-2-(2H-1,2,3-triazol-2-yl)phenyl]methanone;
[5-methyl-2-(2H-1,2,3-azol-2-yl)phenyl][2-(phenylethynyl)piperidin-1-yl 1-{[5-methyl-2-(2H-1,2,3-triazol-2-yl)phenyl]carbonyl}-2-(phenylethynyl)piperidine;
5-fluoro-2-[(1-{[5-methyl-2-(2H-1,2,3-triazol-2-yl)phenyl]carbonyl}piperidin-2-yl)ethynyl]pyridine;
2-[(4-fluorophenyl)ethynyl]-1-{[5-methyl-2-(2H-1,2,3-1xiazol-2-yl)phenyl]2-[(1-{[5-methyl-2-(2H-1_{J}2,3-triazol-2-yl)phenyl]carbonyl}piperidin-2-yl)ethynyl]quinoline;
1-{[5-methyl-2-(2H-1,2,3-triazol-2-yl)phenyl] carbonyl}-2-(naphthalen-2-ylethynyl)piperidine;
(2-merayl-5-phenyl-1,3-thiazol-4-yl) [2-(phenylethynyl)piperidin-1-yl]methanone;
1-[(2-methyl-5-phenyl-1,3-thiazol-4-yl)carbonyl]-2-(phenylethynyl)piperidine;
and pharmaceutically acceptable salts thereof.

In some embodiments, the OX1R antagonist of the present invention is selected from 2-pyridyloxy-3-nitrile-4-substituted orexin receptor antagonists that are disclosed in WO 2014099698 A1. In some embodiments, the OX1R antagonist of the present invention is selected from the group consisting of:
4-(Methylsulfanyl)-2-{[(3R,6R)-6-methyl-1-{[2-(2H-1,2,3-triazol-2-yl)phenyl]carbonyl}-piperidin-3-yl]oxy}pyridine-3-carbonitrile;
4-Methyl-2-{[(3R,6R)-6-methyl-1-{[2-(2H-1,2,3-triazol-2-yl)phenyl]carbonyl}piperidin-3-yl] oxy} pyridine-3-carbonitrile;
2-{[(3R,6R)-1-{[4-Chloro-2-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[4-(2H-1,2,3-triazol-2-yl)pyridin-3-yl]carbonyl}piperidin-3yl] oxy} pyridine-3-carbonitrile;
2-{[(3R,6R)-1-{[2-fluoro-6-(2H-1,2,3-triazol-2-yl)phenyl]carbonyl}-6-methylpiperidin-3-yl]oxy}-4-methylpyridine-3-carbonitrile;
2-{[(3R,6R)-1-{[2-methoxy-6-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[3-methyl-2-(2H-1,2,3-triazol-2-yl)phenyl]carbonyl}piperidin-3-yl]oxy}pyridine-3-carbonitrile;
2-{[(3R,6R)-1-{[4-fluoro-2-(2H-1,2,3-triazol-2-yl)phenyl]carbonyl}-6-methylpiperidin-3-yl]oxy}-4-methylpyridine-3-carbonitrile;
2-{[(3R,6R)-1-{[4-methoxy-2-(2H-1,2,3-triazol-2-yl)phenyl]carbonyl}-6-methylpiperidin-3-yl]oxy}-4-methylpyridine-3-carbonitrile;
2-{[(3R,6R)-1-{[3-fluoro-2-(2H-1,2,3-triazol-2-yl)phenyl]carbonyl}-6-methylpiperidin-3-yl]oxy}-4-methylpyridine-3-carbonitrile;
4-methyl-2-{[(3R,6R)-6-methyl-1-{[5-methyl-2-(2H-1,2,3-triazol-2-yl)phenyl]carbonyl}piperidin-3-yl]oxy}pyridine-3-carbonitrile;
2-{[(3R,6R)-1-{[6-methoxy-2-(2H-1,2,3-triazol-2-yl)pyridin-3-yl]carbonyl}-6-methylpiperidin-3-yl]oxy}-4-methylpyridine-3-carbonitrile;
and pharmaceutically acceptable salts thereof.

The terms “administer” or “administration” refer to the act of injecting or otherwise physically delivering a substance as it exists outside the body (e.g., an OX1R antagonist of the present invention) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiments, the OX1R antagonist of the invention is administered to the subject with a therapeutically effective amount.

By a “therapeutically effective amount” is meant a sufficient amount of OX1R to treat the autoimmune inflammatory disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the OX1R antagonist of the present invention is administered to the subject in combination with a standard treatment.

The OX1R antagonist of the invention is typically combined with pharmaceutically acceptable excipients, and optionally sustained-release matrices, such as biodegradable polymers, to be administered in the form of a pharmaceutical composition. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active antibody in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: OX1R is highly expressed in inflammatory areas of IBD patients but not in the normal colonic mucosa. Left, immunodetection of OX1R in normal colonic mucosa; Middle, immunodetection of OX1R in Crohn's disease (21 samples); Right, immunodetection of OX1R in ulcerative colitis (20 samples).

FIG. 2: OXA ameliorates the DAI (weight and colitis score) of DSS-induced colitis mice. Mice were orally treated with 5% DSS for 7 days in the presence or in the absence of daily intraperitoneal injection of OxA (0.22 μmoles/kg). Left, determination of gain or loss of weight in the absence of DSS treatment (Control,▴), in the presence of DSS treatment (DSS,▪) and in the presence of DSS associated to OxA treatment (DSS+OxA,●). Right, evaluation of Disease Activity Index (DAI) scored by measuring weight, length of colon, diarrhea and blood presence in the stool in DSS untreated control mice (Control,A), in DSS treated mice (DSS,▪) and in DSS treated mice associated to OxA treatment (DSS+OxA,●).

FIG. 3: OXA effects on cytokines secretion in DSS-induced colitis mice. Right, Colons of DSS untreated mice (white), DSS treated mice (gray) and DSS treated mice associated to OxA treatment (black) were resected after animal sacrifice. Then, protein extraction was performed by tissue disruption. Cytokines were determined using Cytokine CBA kits (see Material and Methods). Left, after colon resection, RNA were extracted from colonic tissue and qPCR was performed using specific oligonucleotides for IL8 homolog and IL1B.

FIG. 4: Preventive and curative Orexin A treatments dramatically alleviate EAE symptoms. EAE was induced with 100 μg of MOG₃₅₋₅₅ (Myelin Oligodendrocyte Glycoprotein 35-55) as previously described (Proc Natl Acad Sci USA. 106(6):2012-7, 2009) to 9 week old female C57BL/6 wild-type (WT) mice (n=10/group). Panels show clinical signs of EAE scored daily in a blinded fashion for over 30 days on a scale of 0-5 as follows: 0, no detectable clinical signs, 1, waddling gait with limp tail, 2, ataxia with full paralysis of one limb, 3, full paralysis of two limbs, 4, full paralysis of two limbs with important weight loss (>20% of initial weight) and 5, moribund or dead. Then, mice were given:

Panel A, intraperitoneally (IP) either PBS (group PBS IP), 100 μg of orexin A per mouse for 5 days on day 3 (=before the onset, group BOxA₁₀₀ IP) or at a moderate EAE score (=1,5-2 for group OxA₁₀₀ IP).

Panel B, intraperitoneally either PBS (group PBS), 100 μg (group OxA₁₀₀ IP) or 300 μg (group OxA₃₀₀ IP) of orexin A per mouse for 5 days at a moderate EAE score (=1,5-2).

Panel C, either PBS (group PBS), 300 μg of orexin A per mouse intraperitoneally (group OxA₃₀₀ IP) or retro-orbitally (RO) (group OxA₃₀₀ RO) for 5 days at a moderate EAE score (=1,5-2).

The EAE score is shown as mean+/−SEM. *P<0.05 (nonparametric t-test, compared to PBS group).

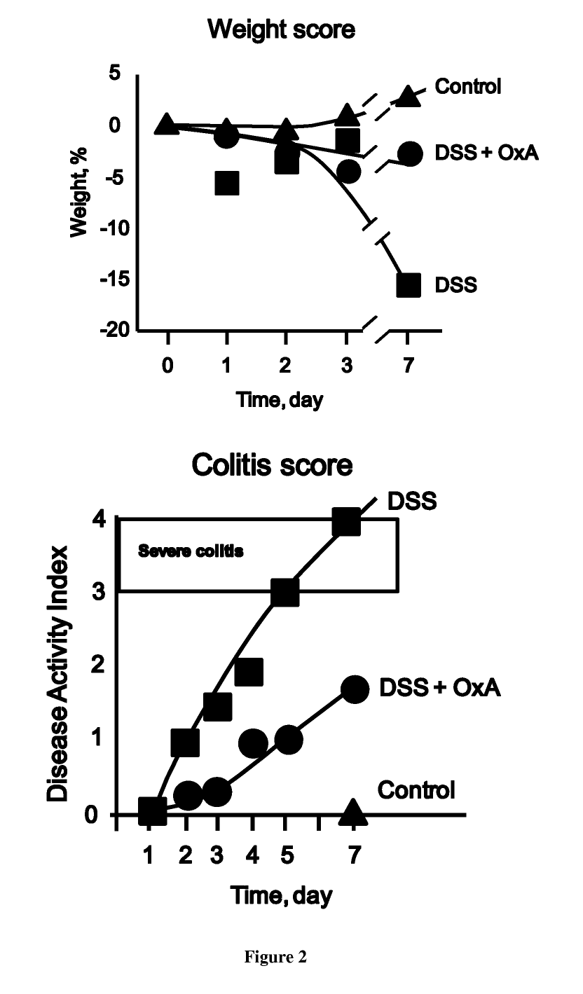

FIG. 5. Orexin A significantly decreases histopathological score of EAE mice. 30 days post-immunization, mice (from the PBS, OxA₃₀₀ RO, and OxA₃₀₀ IP groups of FIG. 2, panel C) were sacrificed and spinal cords were harvested, fixed overnight in 4% paraformaldehyde and stored in 70% ethanol. Spinal cords were then embedded in paraffin and cut in 0.7 μm section. After haematoxylin/eosin (to distinguish cell infiltration) and luxol fast blue (to label myelin) staining, photography was performed at ×1,25 and ×10 magnifications for the spinal cord sections of PBS, OxA₃₀₀ RO and OxA₃₀₀ IP mice. The graph shows the mean scores of each group (n=5/group). Histopathology grading was as follows: 0—normal appearance, 1—some infiltrated cells and low demyelination, 2—2 or 3 infiltrated areas and low demyelination, 3—numerous infiltrated areas and strong demyelination, 4—important cell infiltration throughout the tissue with strong demyelination. *P<0.05 (nonparametric t-test, compared to PBS group).

FIG. 6. Orexin A efficiently suppresses Th1-specific (IFNγ) and Th17-specific (IL-17) cytokine gene expression in the brain of EAE mice. It was determined 30 days after EAE induction of PBS, OxA₃₀₀ RO, and OxA₃₀₀ IP mice (from FIG. 2, panel C) by real time RT-qPCR as previously described (Proc Natl Acad Sci USA. 106(6):2012-7, 2009). *P<0.05 (nonparametric t-test, compared to PBS group).

FIG. 7. Orexin A-treated mice exhibit increased regulatory T cell (Treg) proportion in comparison with PBS-treated mice in draining lymph nodes during EAE. On day 30 post-immunization, draining lymph nodes were harvested from the naïve, PBS, OxA₃₀₀ RO, and OxA₃₀₀ IP groups (FIG. 2, panel C). Assessment by flow cytometry was performed by using the mouse regulatory T cell staining kit: lymph node Tregs were defined as CD4⁺CD25⁺Foxp3⁻ cells and proliferative Tregs as CD4⁺CD25⁺Foxp3⁺Ki67⁺ cells.

Histograms represent the mean of the percentage (left) and the total number (right) of Tregs and proliferative Tregs (Ki67⁺Tregs) for each group. Bars represent the mean+/−SEM of 5 individual mice. *P<0.05 (non parametric t-test, compared to PBS group).

FIG. 8: Scoring of OX1R expression in normal pancreas and pancreatitis in human. OX1R expression was determined by immunohistochemistry using anti-OX1R antibody. Scoring of slices was determined as the intensity of OX1R expression (0 to 3)×the percentage of labeled pancreatitis surface (0 to 100%).

FIG. 9. Pancreatic lymphocyte infiltration determined by immunostaining of CD45+ cells. Values were expressed as percentage of stained surface. **, p<0.01

FIG. 10: Amylase activity in blood samples of control mice, cerulein-induced mice and cerulein-induced mice treated with OxA. *, p<0.05; ***, p<0.001 and NS, non significant.

FIG. 11: Effect of orexin-A and SB408124 antagonist on Ca2+ mobilization in HEK-OX1R cells. Top, HEK-OX1R cells were incubated with fluorescence probe (FluoForte) for 45 min. at 37° C. according to FluoForte calcium assay kit (Enzo Life Sciences). 1 μM of OxA was added to cells and fluorescence emission was measured on TECAN Infinite 200 fluorospectrophotometer. Bottom, HEK-OX1R cells were incubated with fluorescence probe (FluoForte) for 45 min. at 37° C. and then incubated with 1 μM of SB408124 for 1 h at 37° C. After pre-incubation, 1 μM of OxA was added to cells and fluorescence emission was measured.

FIG. 12: Determination of the inhibition of cellular growth of HEK-OX1R cells and colon adenocarcinoma cells (HT-29) induced by 0.1 μM of OxA or various concentrations of SB408124 antagonist. HEK-OX1R cells (black column) and HT-29 cells (white column) were incubated with 0.1 μM of OxA and indicated increasing concentration of SB408124, and cells were counted after 48 hr incubation. Results are expressed as the percentage of total viable cells.

FIG. 13: Effect of orexin-A and SB408124 antagonist on apoptosis in OX1R expressing colon adenocarcinoma cells, HT-29. HT-29 cells were challenged with 1 μM orexin-A or various concentration of SB408124 for 48 h. Apoptosis was measured by determination of annexin V-PE binding, and results are expressed as the percentage of apoptotic cells. Results are means±SE of three experiments. ***P<0.001.

FIG. 14: Effect of daily ip inoculation of OxA, Suvorexant and Almorexant on the length of colon from DSS—(dextran sulfate sodium) treated mice mimicking the acute ulcerative colitis disease. A. Mice were treated with 5% (w/v) DSS in drinking water and daily injected with 20 μg of OxA (OXA IP), 20 μg of Suvorexant (Suvo) or 20 μg of Almorexant (Almo). Control (wt) or treated DSS mice (DSS) were daily injected with 100 μl PBS. After one week of treatment, mice were sacrificed and the length of colon which represented a good marker of inflammation state, was measured. The figure displays a representative experiment. B. quantification of colon length from 5 different DSS-treated mice injected with OxA (DSS+OXA IP), Almorexant (DSS+Almo), Suvorexant (DSS+Suvo) or not injected (WT and DSS). NS, no significant; *, p<0.05; ** p<0.001

### EXAMPLES

### Example 1

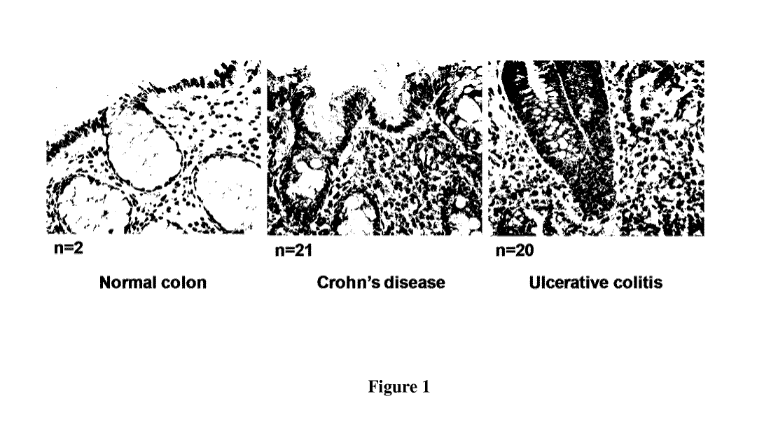

We demonstrate that OX1R was expressed in human Inflammatory Bowel Disease (IBD) including Crohn's disease and UC. Indeed, the use of specific antibodies directed against OX1R in immunohistochemistry (IHC) experiment of about 40 inflamed colonic samples revealed the ectopic presence of OX1R in both epithelial and immune cells (FIG. 1). In contrast OX1R was not expressed in normal colonic mucosa (FIG. 1).

Based on these observations, we have investigated the effect of Orexin A (OxA) on acute inflammation in mice treated with Dextran Sulfate Sodium (DSS). OxA was preferentially chosen in these experiments since the presence of two disulphide bridges confers it more stability. DSS induces acute colitis characterized by weight loss, bloody diarrhea, intestinal ulcerations and infiltrations with granulocytes. Our results indicate that the treatment with OxA in orally DSS-treated mice ameliorates the Disease Activity Index (DAI) scored by measuring weight, length of colon, diarrhea and the presence of blood in the stool (FIG. 2).

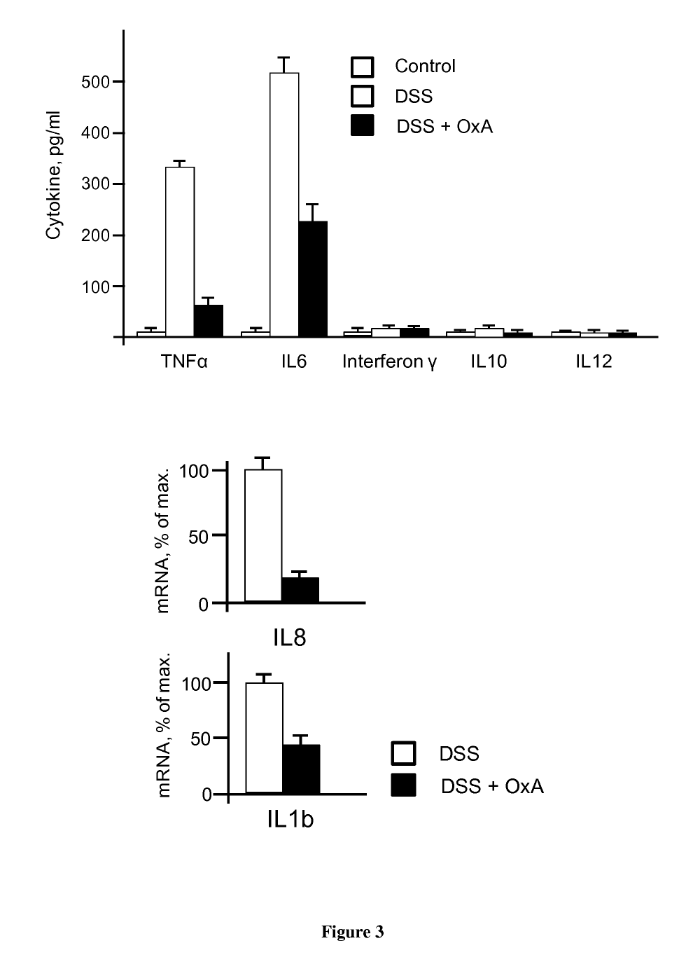

These observations are confirmed by histologic aspect of colon epithelium (histologic scoring by a GI pathologist). Thus, OxA has probably an anti-inflammatory effect on DSS-induced colitis. To confirm this hypothesis, we have investigated the anti-inflammatory effect of OxA treatment on cytokines secretion in colon extracts from DSS-treated mice. The analysis of cytokinic profile revealed that OxA reduces the secretion of “pro-inflammatory” cytokines such as TNFα, IL6, IL8 homolog and IL1B in colon extracts of DSS-induced colitis mice (FIG. 3). In contrast, OxA has no effect on INFγ, IL10, and IL12 cytokine secretion in colon extracts (FIG. 3).

In conclusion these data indicate that OxA could exert an original anti-inflammatory properties in DSS-treated mouse model. Taken into account this proof of concept, the system orexins/OX1R represent an effective target in the treatment of autoimmune inflammatory diseases, in particular ulcerative colitis.

### Example 2

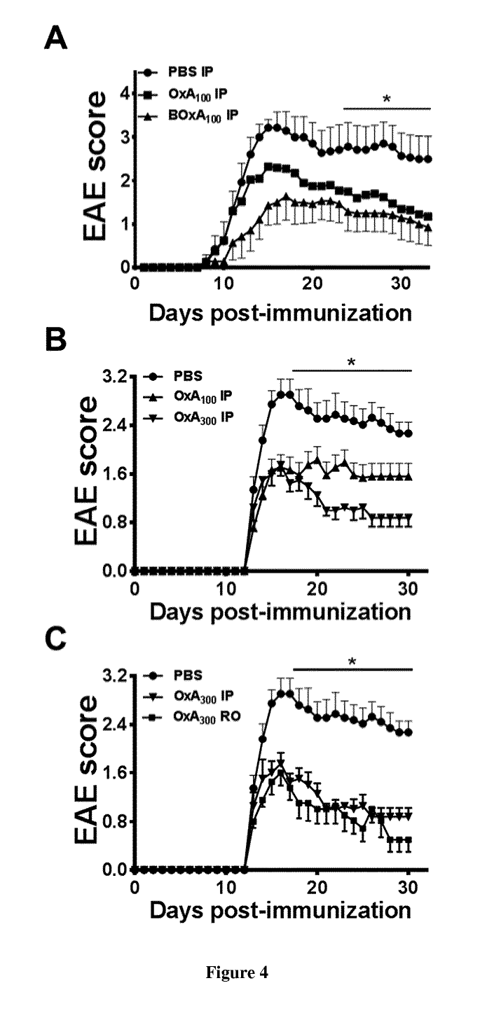

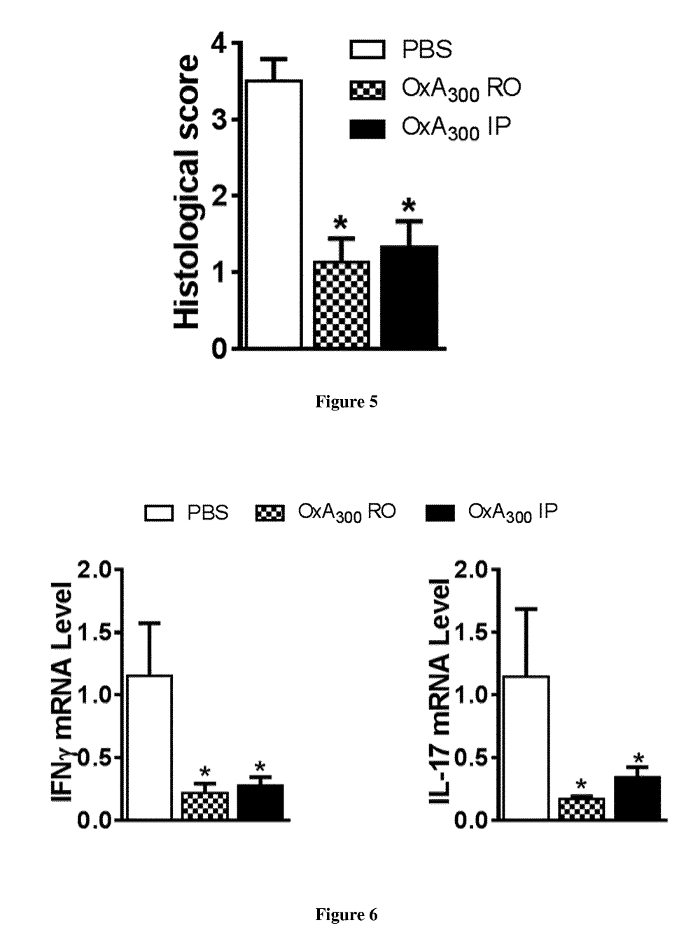

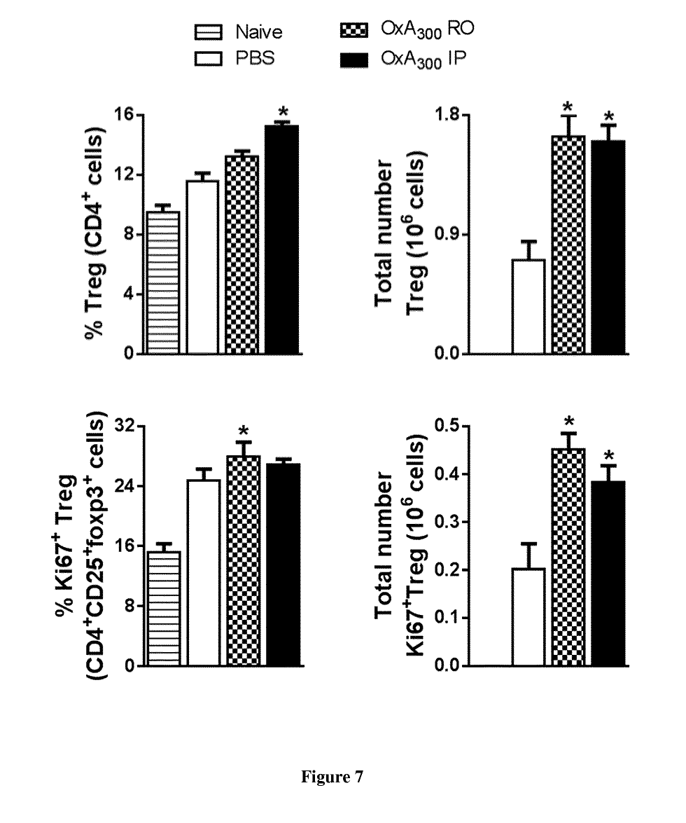

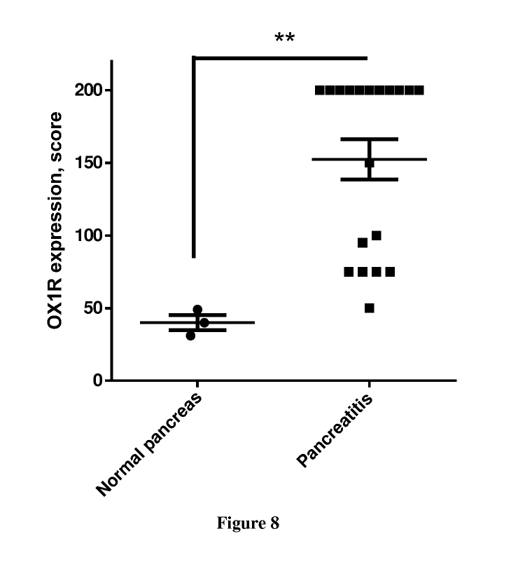

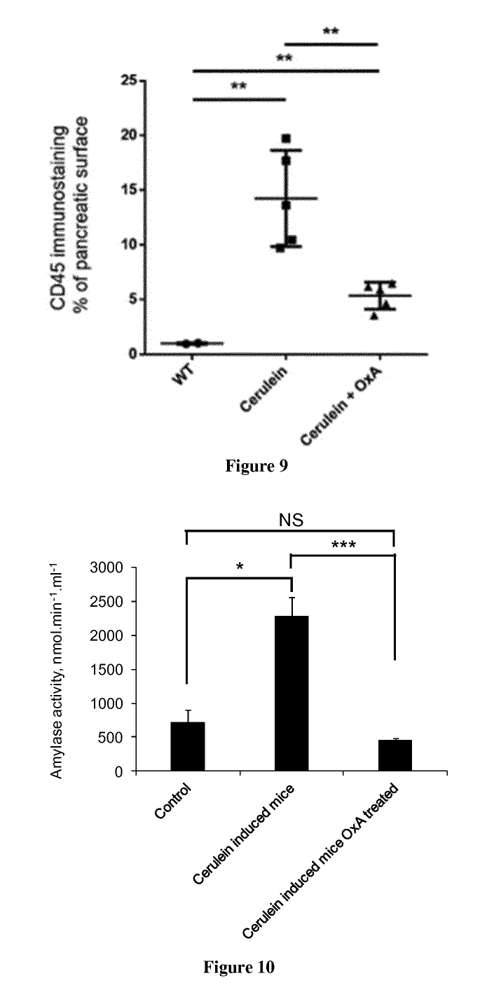

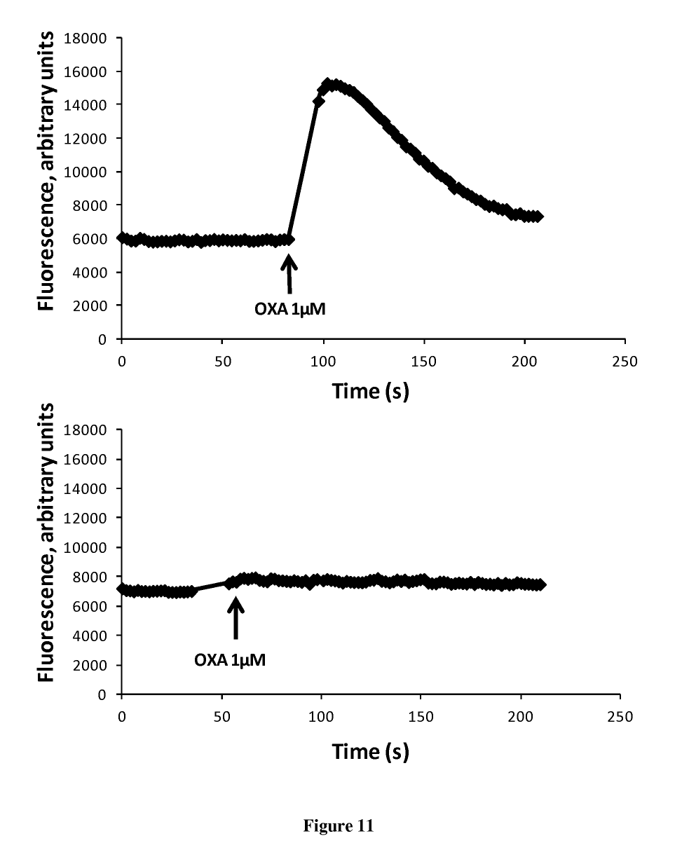

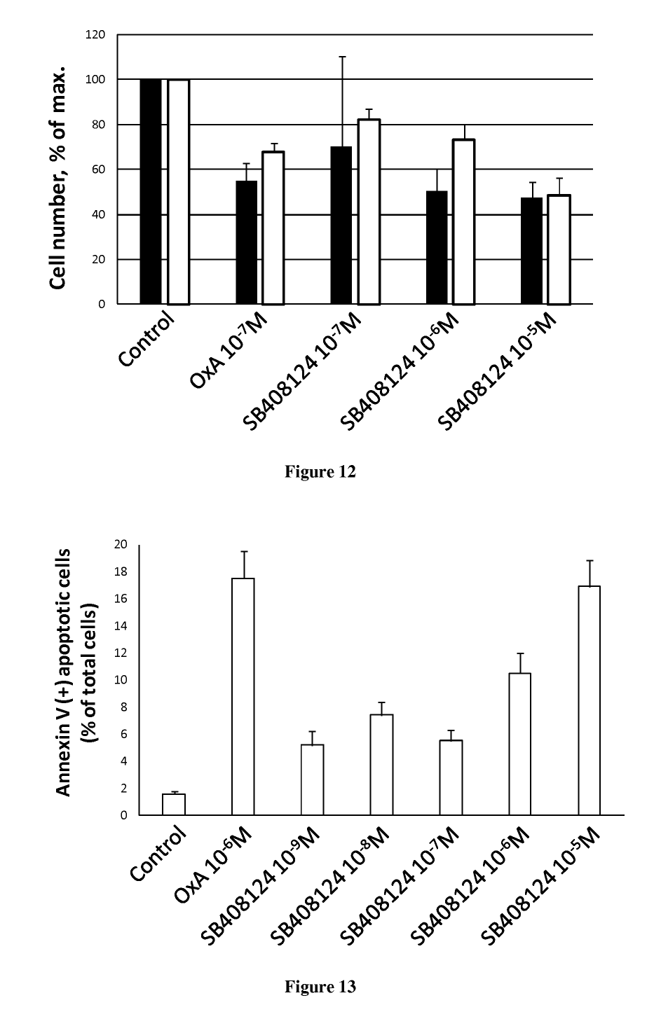

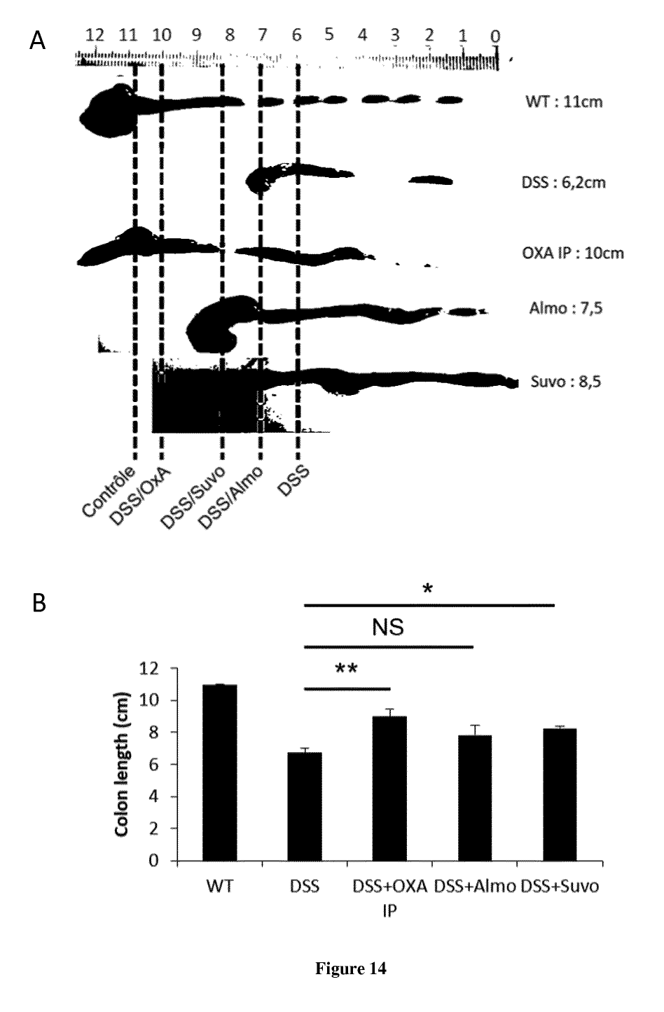

The inventors have found that orexin A administration to mice undergoing chronic experimental autoimmune encephalomyelitis (EAE) (a widely used mouse model for progressive MS) significantly ameliorated the clinical features of the disease at a dose-dependent fashion (FIG. 4). Interestingly, this result was accompanied with drastic reduction of the histopathological EAE score (FIG. 5) and of the Th1/Th17 pro-inflammatory responses (FIG. 6) in the CNS tissues, but with an increase of regulatory T cell (Treg, which play a critical role during inflammation) proportion (FIG. 7) in orexin A treated-mice versus PBS controls. Therefore, orexin A presents potent intrinsic anti-inflammatory properties, capable of modulating the Th/Treg homeostasis during an auto-immune response as aggressive as in a chronic EAE model.

### Example 3

Chronic pancreatitis is a progressive inflammatory disease which leads to the permanent deterioration of the structure and function of the pancreas characterized by inflammation, fibrosis and exocrine/endocrine insufficiency. Orexins (orexin-A and orexin-B) are hypothalamic peptides involved in the sleep/wake control which interact with two GPCR sub-types, OX1R and OX2R. We have recently observed that OX1R is highly expressed in the whole pancreas in human pancreatitis (FIG. 8), whereas it is restricted to islets in the normal pancreas. Moreover, we have demonstrated the anti-inflammatory role of orexin in colitis.

We have investigated the effect of orexin A (OxA) on chronic pancreatitis mice model induced by supraphysiologic doses of cerulein (3 intraperitoneal injections/week at 100 μg/kg). After 3 weeks of cerulein, histological analysis of the pancreas revealed fibrosis, chronic inflammation and acino-ductal metaplasia. In mice treated by OxA (2 intraperitoneal injections/week at 1.40 μmol/Kg) the lesions induced by cerulein improved. OxA-treated mice had lower number of acino-ductal metaplasia. OxA treatment lowered pancreatic fibrosis on Picrosirius staining (12% of the pancreatic surface, as compared to 40% in OxA-untreated mice evaluated by quantitative imaging analysis). OxA treatment reduced pancreatic lymphocyte infiltration evaluated by immunohistochemistry with anti-CD45 antibody (6% of the pancreatic surface, as compared to 18% in OxA-untreated mice evaluated by quantitative imaging analysis) (FIG. 9). Finally the amylase activity is significantly reduced in OxA-treated mice (FIG. 10).

These results demonstrate the protective role of orexin in the development of chronic pancreatitis induced by cerulein in a mice model. In conclusion, the orexins/OX1R system may represents an effective target in the treatment of pancreatitis.

### Example 4

Material & Methods

DSS-induced colitis mice models: Balb/c mice were orally treated by 5% (w/v) of Dextran Sulfate Sodium (DSS) for 7 days. Orexin treatment was carried out in DSS treated mice by daily intraperitoneal injection of OxA (0.22 μmoles/kg) for 7 days. Weight and colitis symptoms (diarrhea, blood in the stool . . . ) for each mouse was daily measured. After 7 days of treatment, animals were sacrificed and colons were resected to further analyses (size, histological aspect, cytokine assays).

Cytokine assays: Mice were sacrificed and colons were resected. Proteins were extracted from colon by tissue disruption in PBS using Tissue Lyser (Qiagen, Courtaboeuf, France). Various cytokines (see FIG. 3) were determined using Cytokine CBA kits (BD Sciences, Le Pont de Claix, France).

Ca2+ mobilization assay: HEK cells expressing recombinant native OX1R (HEK-OX1R) cells were seeded in 96-wells plate, grown and maintained at 37° C. in a humidified 5% CO2/air incubator. 80,000 cells/well were incubated with FluoForte probe according to FluoForte calcium assay kit (ENZO life Sciences, Farmingdale, N.Y., USA) for 45 min. at 37° C. and then incubated with or without 1 μM of SB408124 antagonist for 1 h at 37° C. After pre-incubation, 1 μM of OxA was added and fluorescence was determined using TECAN Infinite 200 fluorospectrophotometer.

Cells growth determination and apoptosis assay: HEK-OX1R cells or colon adenocarcinoma HT-29 cells were seeded, grown and maintained at 37° C. in a humidified 5% CO₂/air incubator. After 24 hr culture, cells were treated with or without Orexin-A peptide or SB408124 antagonist, previously dissolved in DMSO, to be tested at the concentration indicated in the figure legends. After 48 hr of treatment, adherent cells were harvested by Trip1E (Life Technologies, Saint Aubin, France) and manually counted. Apoptosis was determined using the Guava PCA system and the Guava nexin kit.

Results

Without wishing to be bound to any particular theory, it is believed that the results observed in EXAMPLES 1, 2 and 3 are due to the pro-apoptotic effects of Orexin. The inventors have now explored whether the OX1R antagonists of the prior art are capable to induce apoptosis. As shown in FIG. 11, OxA induced a large and transient Ca²⁺ mobilization in HEK-OX1R cells. In contrast, the pre-incubation of cells with 1 μM of SB408124 antagonist totally abolished the induced-Ca²⁺ mobilization (FIG. 11) confirming the antagonist effect of SB408124 on intracellular calcium release mediated by OX1R trough Gq and phospholipase C pathway. In the second phase, we determined the antagonist or agonist effect of SB408124 on cellular growth and apoptosis of HEK-OX1R cells and colon cancer cell line, HT-29. As shown in FIG. 12, OxA induced a strong inhibition of cellular growth of HEK-OX1R and HT-29 cells. Surprisingly, SB408124 antagonist induced also a strong inhibition in a dose-dependent manner of cellular growth of HEK-OX1R and HT-29 cells. As previously shown, orexins were able to trigger an inhibition of cellular growth by induction of mitochondrial apoptosis. As expected, OxA induced an apoptotic effect in HT-29 cells (FIG. 13). Likewise, SB408124 antagonist was also able to induce in a dose-dependent manner cell apoptosis in HT-29 cells. Taken together these results demonstrated that SB408124 was a full antagonist for OX1R-mediated calcium mobilization but a full agonist for OX1R-mediated mitochondrial apoptosis in colon cancer cell line. The inventors now believe that such compounds could be suitable for the treatment of autoimmune inflammatory diseases.

The inventors have thus investigated the effect of daily intraperitoneal inoculation of OxA, Suvorexant and Almorexant in the experimental context of EXAMPLE 1 (colitis). In particular they investigated the effects of the drugs on the length of colon from DSS-(dextran sulfate sodium) treated mice mimicking the acute ulcerative colitis disease. As shown in FIG. 14A and 14B, DSS treated mice show signs of acute ulcerative colitis disease as indicated by the diminution of colon length which represents a good marker of inflammation state. The inventors demonstrated that OX1R antagonists Suvorexant and Almorexant similarly to OxA protect mice from DSS induced acute ulcerative colitis disease (FIGS. 14A and 14B).

In conclusion, considering the teachings of EXAMPLES 1, 2, and 3 showing that Orexin has anti-inflammatory properties and considering the teaching of EXAMPLE 4 showing that OX1R antagonists reproduce the effects of Orexin, it is thus credible to consider that OX1R antagonists such as SB408124 are suitable for the treatment of autoimmune inflammatory diseases. Indeed, these compounds are antagonists for OX1R-mediated calcium mobilization but a full agonist for OX1R-mediated mitochondrial apoptosis, which is the mechanism involved in the improvement of resolution of inflammation observed in the models of colitis, multiple sclerosis and pancreatitis.

### REFERENCES

Throughout this application, various references describe the state of the art to which this invention pertains. The disclosures of these references are hereby incorporated by reference into the present disclosure.

1. A method of treating an autoimmune inflammatory disease in a subject in need thereof comprising administering to the subject a therapeutically effective amount of at least one OX1R antagonist.

2. The method according to claim 1 wherein the OX1R antagonist is a small organic molecule.

3. The method of claim 1 wherein the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

4. The method of claim 1 wherein the autoimmune inflammatory disease is multiple sclerosis.


SEQUENCE LISTING (1 sequence)

SEQ ID NO: 1 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP